(19) United States
(12) Patent Application Publication
(10) Pub. No.: US 2011/0256161 A1
(43) Pub. Date: Oct. 20, 2011

(54) METHODS FOR ENHANCING IMMUNE RESPONSE

(75) Inventors: MARK R. BURNS, KENMORE, WA (US); SUSAN K. GILMOUR, WYNNEWOOD, PA (US); THOMAS G. O'BRIEN, DREXEL HILL, PA (US)

(73) Assignee: AMINEX THERAPEUTICS INC., KENMORE, WA (US)

(21) Appl. No.: 13/090,011

(22) Filed: Apr. 19, 2011

Related U.S. Application Data
(60) Provisional application No. 61/325,544, filed on Apr. 19, 2010.

(51) Int. Cl. A61K 39/00; A61P 35/00; A61P 37/00; A61P 15/18; A61P 31/18; A61P 31/16; A61P 1/04; A61K 31/197; A61P 31/04

(52) U.S. Cl. 424/184.1; 514/564

(57) ABSTRACT

This disclosure demonstrates a novel therapy immunological approach using polyamine-based therapy (PBT) for relieving tumor-induced suppression of the patient's immune system. The demonstration of the pharmacological release from the naturally occurring polyamine-mediated immune suppression offers profound impact on the immunotherapy of cancer together with a variety of diseases caused by the disease-causing vector's ability to evade an immune reaction. This therapeutic approach is equally applicable to disease states whereby immune system suppression by polyamines has been demonstrated including; bacterial infections, parasitic infections including malaria and typanosomiasis, viral infections, peptic ulcers and gastric cancer due to H. Pylori infection together with prevention of pregnancy. With a small molecule drug, used in combination with DFMO, the pharmacological manipulation of polyamine levels for therapeutic benefit in various disease states is possible.

### CROSS-REFERENCE TO RELATED APPLICATIONS

This application claims the benefit of U.S. Provisional Application 61/325,544 filed on Apr. 19, 2010, the contents of which are incorporated herein by reference in their entirety.

### STATEMENT REGARDING FEDERALLY SPONSORED RESEARCH OR DEVELOPMENT

Not applicable.

### FIELD OF THE DISCLOSURE

The disclosure herein relates to the field of pharmaceuticals and medicine and describes the use of a class of polyamine compounds for reducing disease-related suppression of the immune system. As pharmaceuticals, these compounds are useful to treat disorders of undesired cell proliferation (including cancer), infectious disease states (including those due to bacterial or viral infection) or for use as contraceptive agents. By utilizing an underappreciated effect following polyamine depletion of boosting immunological reactions, these agents are useful in the treatment of diseases where a more robust immunological reaction is desired. Examples of such disease states include cancer, bacterial infections or viral infections.

### BACKGROUND OF THE DISCLOSURE

The paradigm of scientific work in the arena of biological systems has historically been to separate and isolate the individual biomolecular particles and targets and test interactions between distinct molecules. The thought is that once the complex nature of mixtures of biological components is reduced, only then can the fundamental biological phenomenon be revealed. This approach has been gloriously successful over the last century of research, with historical improvements in human health and longevity as the demonstrated outcomes. Nevertheless, science is now left with more chronic disease conditions whose complexities outstrip the ability of the isolationist approach to succeed. Looking at any biochemistry molecular chart, e.g., cellular signaling, apoptosis, immunological biomolecules, cytokines, complement or glycobiology features of cell function, one quickly appreciates that these systems are highly redundant, interconnected, interdependent and exceedingly complex. The newer scientific discipline of systems biology aims to understand this complexity, and by definition, must look at ‘whole system’ approaches to comprehend the phenotypical changes occurring in pathological conditions.

While under-appreciated until now, this new approach is especially important in conditions involving infective agents (bacteria, parasites or viruses) together with those indications involving oncogenic changes of the host's cells to a hyperproliferative state (cancer). These disease states involve molecularly-defined interactions between various life forms (or transformed cells actually derived from the ‘host’) that evolved together over eons. Only when the influence of immunology is considered at the level of host-pathogen interactions can the true impact of potential pharmaceutical intervention be observed. This places a high value on animal models where disease occurs in a more ‘spontaneous’ and natural setting. Furthermore, this insight suggests that many missed opportunities have occurred when inherently active pharmaceutical agents might have been tested only against their molecularly isolated targets or in the context of an immunologically inactive model (e.g. athymic or nude mice). Less that optimum effect are observed in these cases. These interventions would therefore be discarded as inactive and not moved forward in the drug development pathway.

Current scientific evidence has highlighted the pathological role that immune evasion plays in major human and animal disease states. By altering the immune response in their presence, transformed cells or microbes and viruses have devised insidious ways to prevent their elimination. Highly aggressive cancer cells with the selected for ability to evade the adaptive and innate immune response have been characterized. Despite the identification and failed therapeutic exploitation of specific tumor-expressed antigens, it is now thought that mechanisms for resistance downstream from the initial T-cell priming may be responsible.¹ It has been shown that a spontaneous anti-tumor T-cell responses can be observed in cancer patients.² The fact that Helicobacter pylori can persist as an infection in its human host for decades highlights this bacterium's ability to abrogate an immune clearance.³ Periodontopathic bacteria exploit an immune system receptor to gain entry into their cellular hosts.⁴ Numerous mechanisms for manipulation of the immune system by cytomegalovirus have recently been reviewed by Sparer.⁵ Various viral functions have evolved to counter natural killer (NK) cell responses to their presence. Viral presentation of protein ligands for expression of regulatory RNA molecules that block NK-activating receptors has been described.⁶ It is beginning to be appreciated that the balance between our immune system's ability to respond to human endogeneous retroviruses (HERV), now known to make up roughly 8% of our genome, has had an important impact on our evolutionary history and can be used to understand the increasing prevalence of certain diseases in our industrialized societies.⁷

The use of chemotherapeutic agents to interrupt cellular metabolic processes constitutes a significant achievement and has supported much advancement in medical treatment over the last half century. As one of the first rationally designed chemotherapeutics, α-difluoromethylornithine (DFMO, FIG. 3a) once held great promise in the fight against cancer.⁸ Despite early results achieved against cancer cells grown in tissue culture, the use of this mechanism-based inhibitor of the first step in the biosynthesis of the polyamines failed to translate into the clinic.^{9, 10} Extensive research now points to the fact that proliferating cells treated with DFMO can overcome this metabolic blockage by importing their required polyamines from extracellular sources. By compensating for the loss of one avenue for obtaining polyamines, the cell utilizes an alternative biochemical mechanism to obtain the molecules necessary for survival and continued growth. Described herein is a method to reduce the levels of polyamines associated with tumors and thereby increase the reactivity of the immune system with the tumor whereby the tumor can be eliminated from the organism.

### BRIEF SUMMARY OF THE DISCLOSURE

This disclosure demonstrates the value of looking at disease states in their nature context thereby freeing these agents to perform their effects within the ‘whole’ system. By using potent pharmacological agents (specifically in this application, a combination of potent polyamine transport inhibitors and polyamine biosynthesis inhibitors), an unexpected mechanism has been uncovered by which the immune system's attack against pathogenic states, including those occurring through generation of hyperproliferative tumor growth, micro-bacterial and viral infections, can be unleashed. The treatment with a combination of a polyamine transport inhibitors and a polyamine biosynthesis inhibitor is herein referred to as Polyamine-Based Therapy (PBT). Furthermore, PBT for the prevention of pregnancy is also possible.

### BRIEF DESCRIPTION OF THE DRAWINGS

FIG. 1 depicts the immuno-editing schematic of cancer hypothesis.

FIG. 2 depicts cellular polyamine metabolism in the context of the whole cell.

FIG. 3 depicts the chemical structure of the exemplary polyamine biosynthesis inhibitor DFMO and the exemplary polyamine transport inhibitors AMXT 1426, AMXT 1569 and AMXT 1501.

FIG. 4 depicts the antitumor effects of the combination of DFMO and AMXT 1501 in the squamous cell carcinoma (SCC) mouse cancer model.

FIG. 5 depicts the antitumor effects of the combination of DFMO and AMXT 1501 in the SCC mouse cancer model.

FIG. 6 depicts the antitumor effects of the combination of DFMO and AMXT 1501 in the SCC mouse cancer model following oral delivery.

FIG. 7 depicts immunohistochemical staining with anti-CD3e antibody of tumor sections following various treatment times with PBT.

FIG. 8 depicts immunohistochemical staining with anti-CD8a antibody of tumor sections following various treatment times with PBT.

FIG. 9 depicts immunohistochemical staining with F4/80 antibody of tumor sections following various treatment times with PBT.

FIG. 10 depicts the increase in level of IFN-γ mRNA following various treatment times with PBT.

FIG. 11 depicts the increase in level of GZMB mRNA following various treatment times with PBT.

FIG. 12 depicts the increase in level of perforin mRNA following various treatment times with PBT.

FIG. 13 depicts an overview of the mechanism of action of PBT.

### DETAILED DESCRIPTION OF THE BEST AND VARIOUS EMBODIMENTS

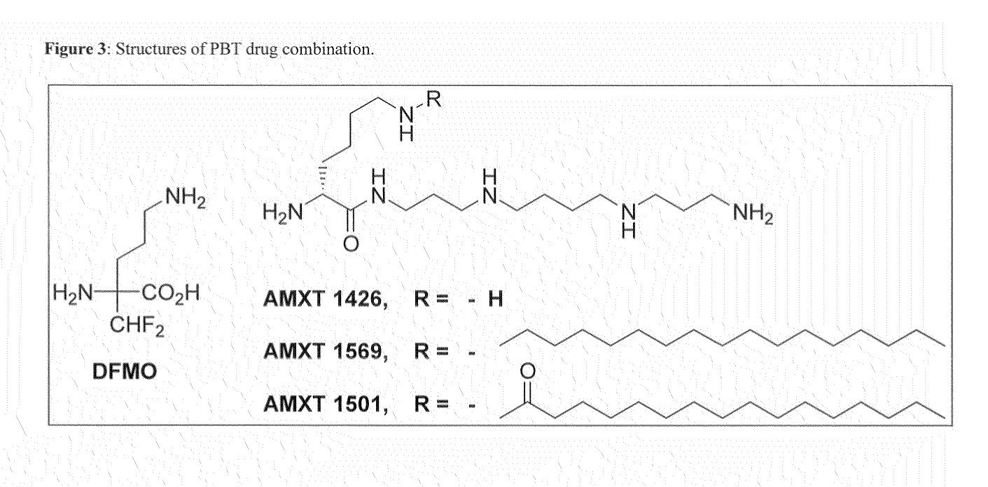

Disclosed herein is a group of lipophilic polyamine analogs (FIG. 3b) that potently inhibit the polyamine uptake system and greatly increase the effectiveness of polyamine depletion when used in combination with DFMO. The resulting novel two-drug combination therapy (named Polyamine-Based Therapy or PBT), which targets cellular polyamine metabolism, has shown exceptional efficacy against a mouse model of squamous cell carcinoma (SCC). A majority (88%) of large, aggressive SCCs exhibited complete or near-complete responses to this combination therapy, while responses to each agent singly were poor. When the tumors that responded to PBT were followed after drug treatment was stopped, it was remarkable to see that the majority of tumors did not re-grow. The availability of these potent polyamine transport inhibitors allows for the clinical development of this molecularly-targeted, first-in-class approach.

Anticancer Effects of Polyamine Depletion. The biology of cancer immuno-surveillance and the mechanism for evasion of tumors from the immunological pressure that has shaped them has molded modern thinking about cancer genesis and immunotherapy treatment failures. The prediction that the immune system represses the growth of cancers was debated from the time it was first proposed by Paul Ehrlich in 1909¹¹ until the first tumor antigen from melanoma was found that was recognized by cytolytic T-lymphocytes (CTLs).¹² Scientific demonstration of the so-called “immuno-surveillance” hypothesis of cancer has been recently revived due to use of mouse tumor models using gene-specific knockout mice. The adaptation of this hypothesis to its current “immunoediting” form redefines the role of host's immune system in reshaping the tumor and has been recently reviewed.¹³⁻¹⁵ In a true Darwinian sense, tumors have evolved a variety of mechanisms for curtailing their reactivity with the immune system and therefore can escape its pressure. It has recently been recognized that conventional chemotherapy and radiotherapy-based cancer treatments must balance their effects on the tumor versus deleterious effects on the immune system's ability to fight the tumor.¹⁶ Indeed, with the present realization that even when primary tumors have apparently been defeated by a combination of therapies, metatheses of tumor or tumor stem cells often times lead to the cancer's reoccurrence. Only when these residual tumors together with their precursor cancer stem cells are eliminated will truly effective and long-lasting treatments be available. Recent progress toward uncovering the means to unleash the immune system and its exquisite specificity to attack these tumors remnants holds great promise for the future of immunotherapy for cancer.

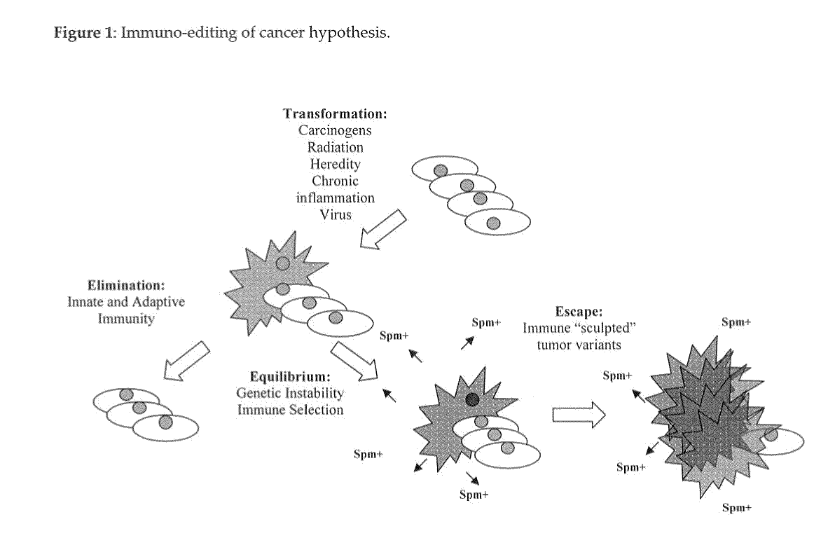

The dynamic interplay between the selective pressures exerted by immunological surveillance and evolutionary adaptations by cancers forms the basis of current thinking about the mechanism of tumor escape from the immune system's watch. The resulting theory predicts a three-phase process involving: Elimination, Equilibrium and Escape stages (FIG. 1). The first phase, Elimination, corresponds to the original immuno-surveillance concept where the immune system is able to eliminate any transformed cells detected. The Equilibrium phase allows the dynamic interplay between the inherent genetic instability of transformed cells to undergo a selection process by the immune system. Those tumors that can counter the constant pressure of immuno-surveillance are selected for survival. The resulting immunologically “sculpted” tumors can enter the third phase of the process, Escape. These tumors have evolved some mechanism for curtailing their reactivity with the immune system and can escape its pressure. Several mechanisms for immunoediting of tumors have been proposed and supported using data from knockout mice without certain components of the IFN-γ signaling pathway, perforin or recombination activating gene 1/2 (RAG-1/2) immune system components.¹⁷

This immunoediting phenomenon has been further supported by evidence in humans of the positive correlation between the presence of CTLs detected in a tumor and increased survival of patients with colon cancer.^{18, 19} Despite the widespread recognition that therapies that utilize the host's immune system should be more effective and less toxic than standard chemotherapy, the success for effective immunotherapy treatments against cancer has only recently been reported.²⁰ By evolving in the presence of the immunological pressure exerted on the tumor, an immunosuppressive bio-molecular network has been generated that protects the tumor from immune attack. A variety of suppressive elements observed in the tumor microenvironment have been described including, 1) predominance of regulatory T cells (T_{reg}) that suppress antitumor effector T cells by producing the immunosuppressive cytokines such as TGF-β or IL-10;²¹ 2) presence of suppressive or dysfunctional dendritic cells;²² or 3) an abundance of suppressive cytokines such as those above and IL-6, VEGF, M-CSF, IDO and others.²³ A variety of human clinical studies have noted that the suppressed immune functions associated with cancer patients can be normalized following removal of the tumor.²⁴⁻²⁶

The presence of high levels of polyamines associated with tumors has been shown to be an alternative mechanism for cancer immune system evasion. Extensive scientific literature referenced herein, together with data given in this invention also supports that polyamine-mediated immune suppression is an additional mechanism by which tumors can accomplish their immune system escape.

Cell growth, especially in hyper-proliferative disease states such as cancer, requires a ready supply of polyamines.^{27, 28} The natural polyamines, putrescine, spermidine and spermine are found in every living cell in high micromolar to low millimolar quantities.²⁸ Increased blood polyamine levels, often observed in cancer patients, have negative impacts on patient prognosis and are associated with tumor progression.²⁹ It is intriguing to note that the increases in blood concentrations of polyamines in cancer patients are dramatically reduced when tumors are removed.²⁹ Furthermore, the induction of the polyamine system, including the biosynthesis enzyme ornithine decarboxylase (ODC) and the transport apparatus, in the hypoxic interior of solid tumors has been shown to result in the increased polyamine concentrations there.³⁰

Decades of research on the myriad of biological activities that the polyamines, putrescine, spermidine and spermine have in cellular processes have shown the profound role they play in life processes.³¹ Chemically in their polycationic form at physiological pH, they tightly bind to and strongly modulate the biological activities of anionic cellular components, including proteins, phospholipids, oligosaccharides and especially nucleic acids.³² It can be concluded that polyamines play important roles in cell proliferation and differentiation. By having powerful pharmacological agents which for the first time control the levels of polyamines, including spermine, it is now demonstrated in this disclosure that the profound effects that polyamines exert on the immune system can be reversed, thereby unleashing the immune system's role in curing diseases.

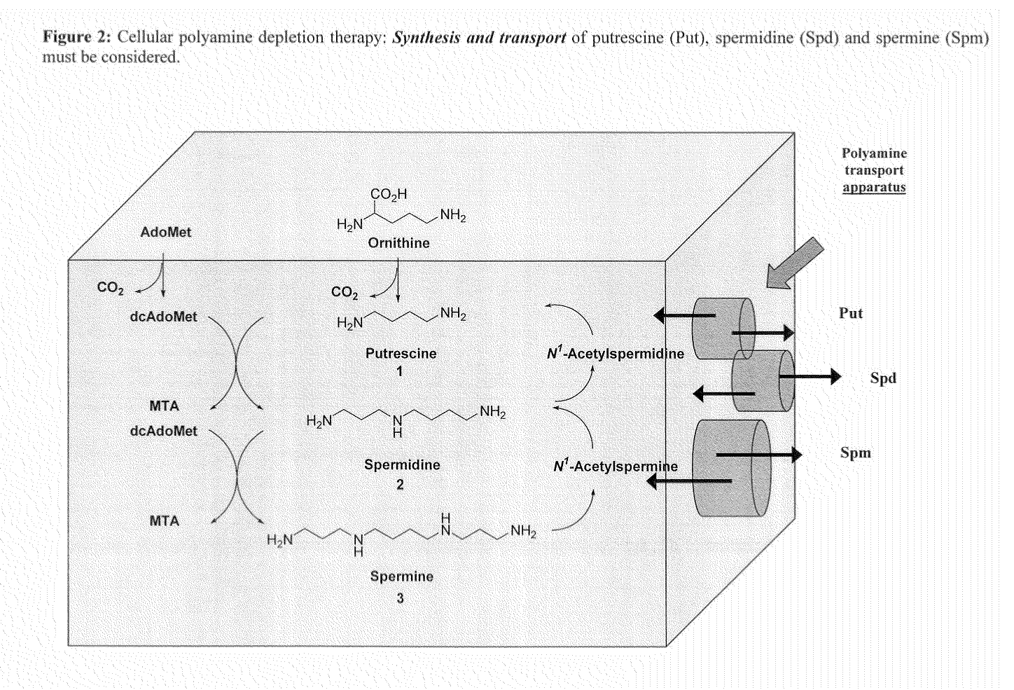

Numerous multidisciplinary studies have shown that intracellular concentrations of polyamines are highly regulated at many steps in their biosynthesis, catabolism and transport (FIG. 2). The presence of such a complex apparatus for the tight control of the levels of these molecules indicates that only a very narrow window of concentrations is tolerated. Ornithine decarboxylase (ODC), the rate-limiting polyamine biosynthetic enzyme, catalyzes conversion of ornithine to putrescine, which is then converted to the tri- and tetra-amines spermidine and spermine. An increase in ODC activity has been associated with tumor growth.³³⁻³⁵ Polyamines are also available to the cell through active transport from the extracellular environment by a transporter located in the cell membrane. Transport of polyamines into mammalian cells is energy and temperature dependent, saturable, carrier-mediated and operates against a substantial concentration gradient.^{36, 37} Ample experimental proof exists that polyamine concentration homeostasis is aided by this transport system. Changes in the requirement for polyamines in response to growth stimulation are reflected by increases in transport activity. Stimulation of human fibroblasts to proliferate by serum or epidermal growth factor leads to an 18-100 fold increase in putrescine uptake.^{38, 39}Furthermore, tumors have also been shown to have an increased rate of uptake of putrescine.^{40, 41} These data strongly imply that polyamine starvation would be an effective strategy to restrict cell proliferation and led to much early enthusiasm for the development of ODC inhibitors such as DFMO.

Some early experiments using DFMO showed that blocking ODC activity produced bone marrow cells that have the capacity to produce significantly more spleen colonies.^{42, 43} The researchers describing these results speculated that effects on the number or function of accessory cells led to increases in the number of hematopoietic precursor cells in mice. It is instructive to note that the thymus of rodents contain one of the higher specific content of the polyamines spermidine and spermine of any tissue in the body.⁴⁴ The spermine concentration in human tissues is fourth highest in the thymus, proceeded by prostate, bone marrow then pancreas as first, second and third.⁴⁵ Furthermore, polyamine levels are found to be high in fetal and neoplastic tissues in addition to seminal fluid; all representing antigenic challenges that fail to elicit an immunological response. Byrd and coworkers found that spermidine and spermine were able to inhibit the induction of immunological responses to phytohemagglutinin, pokeweed mitogen, concanavalin A or bacterial lipopolysaccharide (LPS).⁴⁶ The inhibition observed was dependant upon the presence of calf serum in the media; serum from mouse or human did not work. The authors therefore concluded that spermine or spermidine themselves were not inhibitory; they must be converted into the active inhibitory substance by serum. They went on to suggest that since the inhibition of the immune response could be reversed following washing, these spermidine or spermine degradation products were not acting strictly as toxic agents (i.e. acrolein was not the polyamine-derived inhibitor). The authors raised the suggestion that the product of interaction of polyamine with serum could inhibit immune reactivity in a general way and represent a natural immuno-regulatory agent involved in immuno-suppression observed in fertilization, fetal development and in tumor growth.

Pioneering work by Boon and coworkers demonstrated that a protective immune response can be generated against a non-immune stimulating murine tumor and provided evidence that the tumor's inability to activate the immune system may be due to factors associated with the tumor itself and not its lack of tumor antigens.⁴⁷ Subsequent work has pointed to the role played by immune system regulator and suppressive factors in prevention of clinical exploitation of the precise knowledge of the tumor antigens.⁴⁸ Examples of tumor-specific antigens include gene-encoded products such as MAGE, BAGE or GAGE that are silent in most normal tissues but are expressed in a large proportion of melanomas, lung tumors, head and neck tumors and ladder carcinomas.⁴⁹

Several studies have demonstrated an immunological inhibitory effect of increased levels of polyamines surrounding tumors. Moulinoux and coworkers described experiments where a complete depletion of polyamine levels in mice grafted with 3LL (Lewis lung) carcinoma was accomplished by treatment with DFMO, a polyamine oxidase inhibitor and neomycin to prevent the gut microbrial flora from providing polyamines. In these mice, tumor growth was reduced and immune system abnormalities seen in tumor-bearing animals were reversed.⁵⁰ The decreased spleen cell interleukin 2 (IL-2) production and CD4+ and CD8+ lymphocyte populations observed prior to treatment with drugs were reversed and previously increased polyamine levels in the spleen were lowered. It was necessary to maintain a total blockage of all major polyamine sources to see these reversals. The T-lymphocyte population restoration did not depend upon the stage of tumor growth. No other vaccine activation or tumor-directing antigens were required. It was therefore demonstrated that complete polyamine deprivation reduces tumor-induced immune suppression.

Additionally, Moulinoux and coworkers examined the effects of more total polyamine depletion in mice grafted with 3LL carcinoma in relation to the re-stimulation of the non-specific immune system specializing in tumor cell killing.⁵¹ The dramatic decrease in the cytotoxic activity of their natural killer (NK) cells is reversed in these polyamine depleted animals. The authors conclude that polyamines, secreted by the tumor itself as well as absorbed through the gastrointestinal tract, can be considered not only as autocrine growth factors but also as natural immunosuppressive factors.

Soda and coworkers studied the effects of polyamines on cellular immune function.⁵² Peripheral blood mononuclear cells (PBMCs) from healthy volunteers were cultured with spermine, spermidine or putrescine and the results on immune cell function were examined. Treatment resulted in decreased adhesion of non-stimulated PBMCs to tissue culture plastic in a dose- and time-dependent manner without affecting cell viability or activity. This decreased adhesion was also associated with a decrease in the number of CD11a positive and CD56 positive cells. In a group of 25 cancer patients, changes in blood spermine levels after surgery were negatively correlated with changes in lymphokine-activated killer cells (LAK) cytotoxicity. These authors concluded that increased blood spermine levels maybe an important factor in the suppression of anti-tumor immune cell function.

A study reported by Bowlin noted the effect of the polyamine biosynthesis inhibitor DFMO on immune system cell expression in normal and tumor-bearing (B16 melanoma) C57BL/6 mice.⁵³ DFMO treatment of these immune competent mice for 6 days reduced splenic leukocyte polyamine levels and resulted in the induction of cytotoxic T-lymphocytes in both normal and tumor-bearing animals. While putrescine and spermidine levels were significantly reduced, spermine levels were not. This led the authors to suggest that the generation of CTLs is sensitive to spermine levels. Another study by the same authors explored the effect of treatment by each of three different ornithine decarboxylase inhibitors on tumoricidal macrophage activities in vivo.⁵⁴ Tumor bearing mice that were treated with 0.5 to 2.0% oral DFMO had two-fold augmented macrophage mediated cytolysis of B16F1 cells ex vivo. The authors speculate that the immune sensitivity of a particular tumor may be an important component regarding its sensitivity to ODC inhibitors. An earlier study by Bowlin showed that polyamine oxidation down-regulates IL-2 production by human peripheral blood mononuclear cells.⁵⁵

Gensler reported studies exploring the ability of DFMO to prevent skin carcinogenesis and immunosuppression induced by ultraviolet irradiation in immuno-competent BALB/c mice.⁵⁶ Mice pretreated for 3 weeks with 1% DFMO in their drinking water and then irradiated with UVB radiation had a reduced, 9% occurrence of skin cancer whereas the untreated control group developed cancers in 38% of the mice. The degree of removal of immunosuppression in the DFMO-treated mice was measured by a passive-transfer assay. Splenocytes from UV-irradiated mice when transferred to naïve mice prevented their normal ability to reject UV-induced tumor challenges (20 of 24 of mice grew tumors). When the splenocytes from UV-irradiated mice that where treated with DFMO were transferred to naïve mice, the majority of tumors were rejected (only 2 of 24 grew). This study demonstrated that immunity from UV-induced tumors could be dramatically increased by treatment with the polyamine biosynthesis inhibitor DFMO. Furthermore, these studies demonstrated that an active immune reaction to UV-transformed cells could be transferred from one mouse to another, without the need for some type of vaccine-like specificity-directing component.

Studies in human cancer patients also support the role of polyamines in immune system modulation. Gervais reported experiments looking at the phenotype and functional activity of dendritic cells from cancer patients and investigated the effect of putrescine on these immune cells. Cells from cancer patients yielded a lower yield of dendritic cells and these cells showed a weaker expression of MHC class II molecules. By adding putrescine to dendritic cells from normal donors, it was possible to reduce the final cytolytic activity of lymphocytes, mimicking the defective dendritic cell function of cancer patients.⁵⁷ Evans demonstrated that spermine suppresses the sensitivity of cervical carcinoma cells to cytotoxic LAK lymphocytes collected from more than half the human subjects studied.⁵⁸ The authors suggest that spermine may be an important immunosuppressive agent in natural immunity against cervical cancer.

Tracey has reported that spermine has an immune inhibitory effect.⁵⁹ Specifically, Tracey demonstrated that LPS stimulation of monocytes causes a significant increase in the uptake of spermine by the polyamine transport apparatus of the cell. They used a polyamine transport inhibitor, 4-bis(3-aminopropyl)-piperazine (BAP) (with much lower potency compared to AMXT 1501) to block the inhibitory activity of spermine on monocyte TNF production. This experiment showed that spermine uptake into monocytes is needed to suppress immune function. Experiments using carrageenan-induced inflammation in rats also showed BAP enhanced the production of TNFα and increased the resulting edema in the foot pad.⁶⁰ Additional studies demonstrate that polyamines invoke a suppression of immune system attack on tumor cells.^{61, 62} From these studies it is apparent that polyamines, especially spermine, are involved in attenuating the immunological attack on tumors.

Szabo and colleagues reported studies exploring the mechanism of inhibitory effect of polyamines on the induction of nitric oxide synthase (NOS). They demonstrated the need for the serum-mediated oxidation of spermine to produce its dialdehyde product (called SDA) for immune system inhibition to occur.⁶³ Casero and Wilson reported their work detailing the ability of spermine to inhibit the production of the macrophage-derived NO coming from the inducible NO synthase (iNOS).⁶⁴ The NO produced by the enzyme iNOS is a central effector molecule in the innate immune response to pathogens and is the focus of many groups working towards understanding the role of the microbe H. pylori plays in the pathogenesis of stomach ulcers and gastric cancer. Their evidence supported a mechanism where spermine (but not its oxidation products) potently inhibits iNOS protein translation. With IC₅₀ values of 9.2 μM in RAW 264.7 cells and 9.0 μM in peritoneal macrophages, spermine was demonstrated to have a strong effect at the level of iNOS protein translation. Their evidence furthermore points to the importance of spermine but not putrescine or spermidine as the main mediator of this iNOS inhibition.⁶⁵

DFMO can lower the cellular concentration of putrescine and spermidine but in many cases has been reported to raise the level of spermine. It is thought that this may be due to compensatory increased levels of dcAdoMet that facilitate the metabolic production of spermine in the absence of adequate polyamine precursors. Additionally, an polyamine-level feedback control-mediated increase in the level of the enzyme AdoMet decarboxylase is observed following DFMO treatment.⁶⁶ Casero and Wilson reported that treatment of H. pylori-stimulated RAW 264.7 cells with DFMO resulted in decreased putrescine and spermidine levels and in increased spermine levels. They reported a parallel inhibition of iNOS protein expression and NO production. This result points to the need to ensure pharmacological reduction in spermine levels in order to fully overcome the polyamines' inhibitory effect on the immune system. Further studies by Wilson demonstrated that the induction of ODC by H. pylori contributes to the persistence of the bacterium. The inverse correlation between macrophage-generated NO levels and bacteria levels together with data showing ⁱNOS −/− macrophages failed to kill H. pylori and that iNOS −/− mice infected with H. pylori have increased bacteria colonization and gastritis severity all support the connection between polyamines and immune suppression.⁶⁷ Therefore, the use of PBT to effectively lower the spermine levels in stomach mucosa and H. pylori activated macrophages would be an effective methodology to control gastric cancer or ulcers. Facilitating the immune system's attack of this microbe would allow the clearance of this infection followed by healing of the ulcers and gastric cancers.

Bowlin and coworkers described the inverse correlation between the production of the immune-activating IL-2 cytokine and the concentration of polyamines in rheumatoid arthritis synovial fluid mononuclear cells.⁶⁸ These workers also reported that IL-2 production by normal and rheumatoid arthritis peripheral blood mononuclear cells is down-regulated by products of polyamine metabolism.^{69, 55} Data is also presented that treatment with inhibitors of ornithine decarboxylase increases IL-2 levels. Furthermore, polyamine oxidase (PAO) inhibitors and catalase also increased IL-2 production. Therefore, polyamines and their oxidation products downregulate IL-2 production and may account for the decreased T-cell effector function seen in rheumatoid arthritis.

Additional studies support Bowlin's immunological basis of action of polyamine analog anticancer agents acting through PAO and subsequent generation of H₂O₂. Immunocompetent C57BL mice with L1210 leukemia inoculations were cured following treatment with the spermine analog N,N′-bis[3-(ethylamino)-propyl]-1-7-heptane diamine (BEPH).⁷⁰ It was remarkable that when these cured mice were challenged a second time with L1210 tumor cells they were immune to development of tumors. It was interesting to note that the immune reaction was specific for tumor type as mice subsequently challenged with P388 leukemia cells were not cured. This result is highly suggestive of the exposure of some type of tumor-type specific antigen by this polyamine analog. Transplantation of splenocytes from cured mice with L1210 cells into naïve mice generated a potent tumor-specific cytolytic activity. Treatment of these splenocytes with anti-Thy-1.2 monoclonal antibodies and complement demonstrated a T-cell mediated immune response. In T-cell deficient nude mice BEPH treatment was not curative. These studies demonstrated a pivotal role for T-cell mediated immunity in the anticancer effects of this polyamine analog. While BEPH does have a direct antitumor activity, these studies showed that development of antitumor immunity in BEPH-treated mice facilitates the therapeutic effects of this drug. At the doses used, BEPH had no effect on tumor or spleen cell polyamine levels⁷¹ suggesting that this spermine analog may be competing for the nature polyamine and blocking its immunosuppressive activity.

A similar series of experiments were reported by Umezawa using sperqualin, an antitumor antibiotic structurally related to spermine.⁷² Immunocompetent BALB/C×DBA/2 F1 mice inoculated with L1210 cells survived more than 60 days when treated with sperqualin (i.p. 5 mg/kg for 9 days). These cured mice rejected a second inoculation of L1210 leukemia cells but not P388 cells. The cytotoxic effects of spleen cells, like the study reported by Bowlin, were abrogated by prior treatment with anti-Thy-1.2 antibodies and complement. The antitumor activity was much lower in T-cell deficient athymic mice. These studies suggest that cytotoxic T-lymphocytes are involved in the antitumor action of sperqualin.

Alternative mechanisms might also be responsible for the observed immune inducing effects of PBT. The FDA-approved compound, plerixafor (also known as AMD3100), is used as a hematopoietic stem cell mobilizer for use in combination with granulocyte-colony stimulating factor (G-CSF) for peripheral blood collection and subsequent autologous transplantation in patients with non-Hodgkin's lymphoma and multiple myeloma. This drug has been shown to be an inhibitor of the CXCR4 chemokine receptor's interaction with its ligand, stromal cell-derived factor-1a (SDF-1α, also known as CXCL12).⁷³ Inhibitors of CXCR4/SDF-α interaction have been explored as anticancer agents and have been shown to inhibit the initial proliferation and survival of cancer cell metastases. A report by Luker⁷⁴ showed that through the use of either RNAi knockdown of CXCR4, or treatment with AMD3100, the growth of murine 4T1 breast cancer cells transplanted into mice was significantly reduced. Metatheses of these tumors did not occur in the treated animals and it was remarkable to observe that inhibition of the interaction of CXCR4 with its ligand totally prevented tumor formation in some animals. We tested the effect of 10 μM AMXT 1501 on the binding of radiolabeled SDF-1α to the CXCR4 receptor and found no inhibition. It is important to mention here that testing of AMXT 1501 alone in the K6/ODC SCC murine tumor model did not reduce tumor growth, demonstrating that a synergistic role between AMXT 1501 and DFMO exists. Since an interaction between AMXT 1501 and the CXCR4 receptor was not observed, a polyamine-dependant mechanism of immune-suppression is maintained.

A small stimulatory effect on human TLR 2 and TLR4 was observed when AMXT 1501 was tested at 10 μM concentration in a Toll-Like Receptor (TLR) screening assay.⁷⁵ Relatively low affinity to these innate immune pattern recognition receptors (19% of control using HKLM on TLR2 and 16% of control LPS on TLR4) in comparison to 100× higher affinity implicated for the drug's binding to the polyamine transporter were measured. There was no significant activity of AMXT 1501 on human TLR3, TLR5, TLR7, TLR8 or TLR9. This data suggests that an innate immune effect operating through the TLR system is not part of the mechanism of action of PBT treatment.

Anti-Infective Effects of Polyamine Depletion. The antiviral and antibacterial effects following pharmacological polyamine depletion have only been sporadically reported in the scientific literature. When these reports are viewed in light of the positive effects that polyamine depletion has on the function of the host's immunological reaction to the infection, the pharmacological anti-infective behavior of these agents is apparent.

Bitonti and coworkers reported experiments showing the necessity of an antibody response in the treatment of African trypanosomiasis with DFMO.⁷⁶ Immune suppression of rats using dexamethasone resulted in lower production of a trypanosome-specific antibodies, an impaired ability of the animals to eliminate the infection when given normally curative doses of DFMO and an inability to produce cured animals. It is evident that one of the consequences of a natural trypanosome infection in humans,⁷⁷ as well as experimental infections in animals, is the suppression on both cell-mediated and humoral immunity.⁷⁸ Additional studies, reported using DFMO in a mouse model of Plasmodium berghei malarial infection, showed that a protective immunity against this parasite was produced.^{79, 80} In these studies, mice inoculated with P. Berghei malaria sporozoites, while being treated with DFMO, developed a protective immunity against subsequent challenges with the parasite. It was noted that this protection was long-lasting (at least six months) but was not completely effective in all the mice analyzed. It was suggested that this drug was not effective against the erythrocytic schizont form of the parasites. Given our data showing the ability of our polyamine transport inhibitors to stop the uptake of polyamines into cells in culture, it is expected that their use in combination with DFMO would be an effective treatment against malaria in humans. Furthermore, because DFMO itself was able to induce a immunity against the malarial parasites in mice, its use in combination with a polyamine uptake inhibitor is expected to potentiate its ability to induce an immunological response to the parasite. This will lead to an effective, and long-lived, therapy for malaria.

The activity of polyamine biosynthetic enzymes and the levels of polyamines have been shown to be increased following infection of MRC-5 cells by cytomegalovirus.⁸¹ Furthermore, an increased uptake of radiolabeled putrescine into CMV infected MRC-5 cells was reported.⁸² This research group also described the limited effectiveness of the use of DFMO or methyglyoxal bis(guanylhydrazone) for inhibiting the replication of herpes simplex virus type 1 or herpes simplex virus type 2 but did demonstrate these polyamine biosynthesis inhibitor's effectiveness against CMV infectivity of MRC-5 cells.⁸³ Spermine levels where increased two to ten fold in fibroblast cells infected with the Colburn strain of HCMV.⁸⁴ Lymphocytes isolated from HIV patients have elevated levels of all three polyamines.⁸⁵ Increased polyamine levels are also observed in cells infected with Rous sarcoma virus.^{86, 87} Secrist and coworkers demonstrated an increased level of polyamines but showed that treatment of an HIV-1 infected human T-lymphocyte cell line (CEM) with DFMO had only a moderate block of virus-induced cytopathic effects.⁸⁸ These researchers took these results, obtained in the absence of an immune system in their in vitro system, and concluded that polyamine biosynthesis inhibitors would not be therapeutically useful for the treatment of AIDS. Despite the implied lack of a functioning immune system in the pathology of AIDS, treatment of patients with an immune-system stimulating chemotherapeutic is still warranted. Most HIV-positive patients that are treated with modern antiviral agents retain a functioning T-cell population and are therefore candidates for PBT treatment.

Gibson and coworkers followed up some early results using DFMO as an antiviral agent in their studies describing this agent's effectiveness against cytomegalovirus (CMV). ⁸⁹ They concluded that this inhibitor of polyamine biosynthesis showed strong antiviral activities but only when added before infection of cell culture (human foreskin fibroblast cells).

Contraceptive Effects of Polyamine Depletion. The contra-gestational effects of DFMO have been reported in hamsters,⁹⁰ rats⁹¹ and rabbits.⁹² Factors associated with seminal fluid were identified by Valley to potently suppress natural killer cell activity and this factor's identity was shown to be a polyamine.⁹³ Lea and coworkers reported that higher levels of spermine in in vitro fertilization culture supernatants were predictive of failure to establish pregnancy.⁹⁴ Fernandez reported on the role of polyamines and their oxidases in the etiology of human cervical cancer.⁹⁵ They suggested that the immunosuppressive role of spermine and its oxidation products, especially when combined with the anti-apoptotic effect of HPV infections, could contribute for the survival and proliferation of transformed cells in the cervix.

Recent results suggest the immune system of the mice is playing a role in the dramatic antitumor results seen following treatment with PBT. The immunocompetent FVB/N mice that were used in these studies, in contrast to nude mice used in typical xenograft studies, appear to mount an immunological attack on the tumors. With treatment, an increased level of CD3e⁺ and CD8a⁺ cytolytic T-lymphocytes together anti-F4/80 antibody labeled infiltrating macrophages were detected by staining tumor sections. This disclosure demonstrates that the reduction of the levels of polyamines associated with tumor cells, and, thus, the suppression of the immune system by the tumor itself is greatly reduced.

These results suggest profound implications for the future of immunotherapy against cancers. While extensive prior literature associated a role played by the increased levels of polyamines in the suppression of the immune system's response to tumors, no small molecule pharmacological agents have been available to accomplish this in an animal model. In an additional animal model, PBT was tested using oral dosing in domestic cats with histologically confirmed oral SCC, with positive results. The demonstration of efficacy using this spontaneously generated animal tumor model has been noted to be highly predictive of a favorable outcome in the human clinical setting.⁹⁶

Cancer cells can circumvent the ability of drugs such as α-difluoromethylornithine (DFMO) based on inhibition of polyamine biosynthesis, from completely depleting their internal polyamines by the importation of these molecules from external sources. Described herein is the development of a group of lipophilic polyamine analogs that potently inhibit this polyamine uptake system and greatly increase the effectiveness of polyamine depletion when used in combination with DFMO, even in the presence of extracellular polyamine.⁹⁷ By the attachment of an optimized length C₁₆ lipophilic substituent to the ε-nitrogen atom of our earlier lead compound, D-Lys-Spm (AMXT 1426), we have produced an analog D-Lys(C₁₆acyl)-Spm (AMXT 1501) with several orders of magnitude higher potency against a variety of cultured cancer cell types (including MDA-MB-231, PC-3, A375 and SK-OV-3 among others). By all indications, the cell culture effects appear to be cytostatic and not cytotoxic. The resulting novel two-drug combination therapy targeting cellular polyamine metabolism has shown exceptional efficacy against cutaneous squamous cell carcinomas (SCCs) in a transgenic ornithine decarboxylase (ODC) mouse model developed by us for the study of skin cancer. A majority (88%) of large, aggressive SCCs exhibited complete or near-complete responses to this combination therapy, while responses to each agent singly were poor. The availability of a potent polyamine transport inhibitor allows, for the first time, for a real test of the hypothesis that starving cells of polyamines will lead to objective clinical response. This therapy is molecularly-targeted, relatively nontoxic at the proposed dose, and consists of both a very well-characterized drug and a member of a novel chemical class. Our results showed a strikingly increased infiltration of CD3⁺ and CD8⁺ cytolytic T-lymphocytes in treated tumors. Additionally, a 25× and 40× fold increase in the immunologically important cytokines IFN-γ and GZMB mRNA levels were noted in treated tumors.

The combination therapy of polyamine biosynthesis/uptake inhibition was tested against the K6/ODC transgenic mouse murine squamous cell carcinoma (SCC) model recently described.⁹⁸ The K6/ODC model was developed to assess whether ODC overexpression was a contributing cause, or an effect of malignant transformation. Using a bovine keratin 6 (K6) promoter to drive high-level ODC expression specifically in hair follicles (where presumed targets of carcinogens reside), we were able to demonstrate skin tumor development after only a single low dose of the carcinogen 7,12-dimethylbenz-(α)-anthracene (DMBA), as compared with non-transgenic mice of the same strain that did not show significant tumorigenesis in response to the same treatment. While most skin tumorigenesis models yielded benign squamous papillomas as the predominant tumor type, when the K6/ODC transgene was expressed on the FVB/N strain background, the majority of tumors that developed were aggressive squamous cell carcinomas. These SCCs appeared as early as 5 weeks after treatment and in high multiplicities (up to four tumors per mouse), making this a very efficient model for SCC induction. Using this model, we were thus able to conclude that over-expression of ODC is a sufficient condition for tumor promotion in mouse skin.⁹⁹

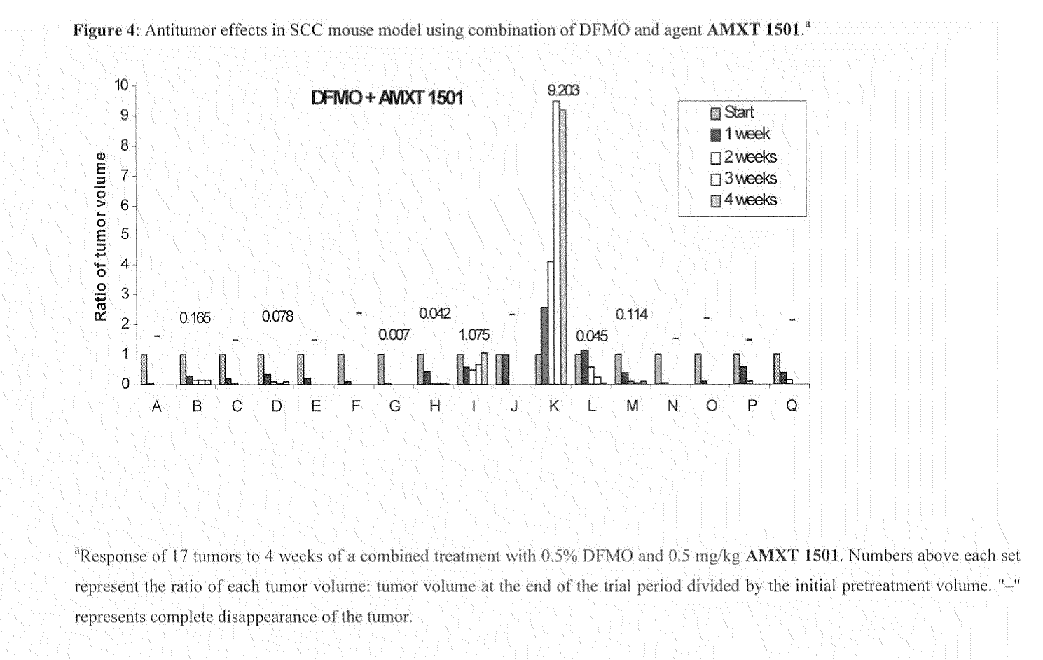

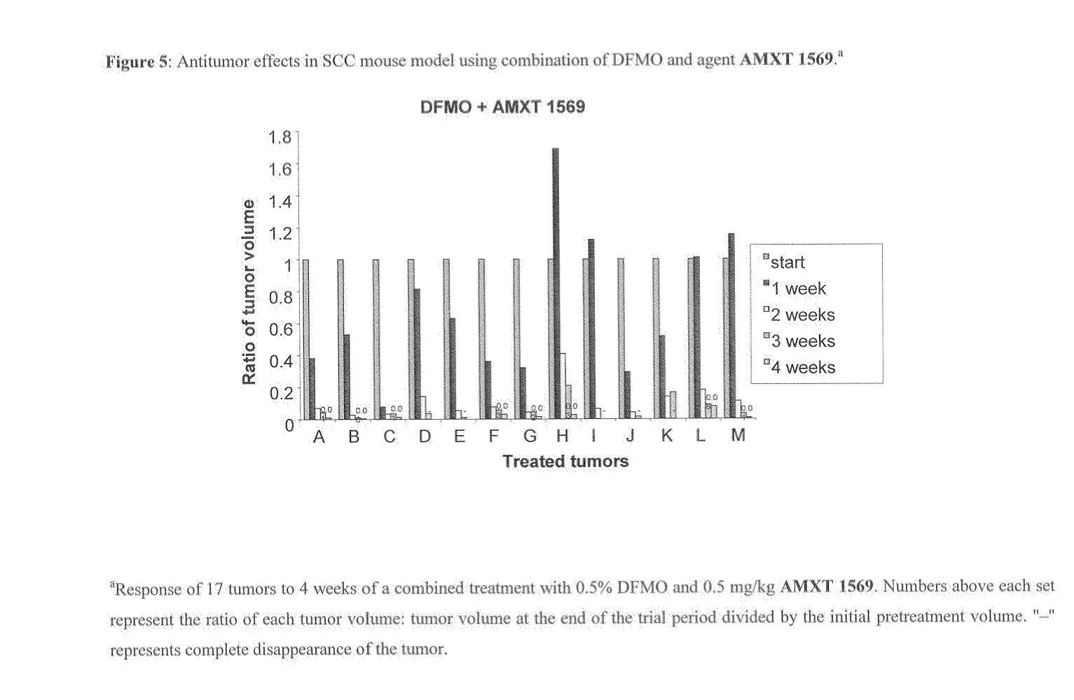

We conducted an in vivo anti-tumor trial of DFMO combined with either the D-C₁₆-acyl Lys-spm conjugate AMXT 1501 (FIG. 4) or the L-C₁₆-alkyl AMXT 1569 analog (FIG. 5) in the K6/ODC SCC model. The potency of these agents to inhibit tumor growth in combination with DFMO corresponded to their relative activities in tissue culture with slightly better results observed using AMXT 1501 in the combination. Comparable efficacy of the DFMO/AMXT 1501 combination was achieved at a 100-fold lower dose compared to our earlier described compound AMXT 1426 (D-Lys-spm, 100 mg/kg/d vs. 1.4 mg/kg/d). The combination of DFMO (at 0.5% in drinking water) and 0.5 mg/kg AMXT 1501 (i.p. twice daily) caused most SCCs (88%) to exhibit complete or near-complete responses (>95% volume reduction), in contrast to the weak effect of DFMO alone. Furthermore, when the 9 out of 17 SCCs that exhibited complete responses were followed for an additional 6 weeks off-treatment, only one tumor recurrence was observed. Based on these results, obtained using a 100-fold lower dosage level of AMXT 1501 compared with our earlier lead, and with no apparent toxicity at this dose level, we feel that clinical development of AMXT 1501 instead of AMXT 1426 holds much greater promise.

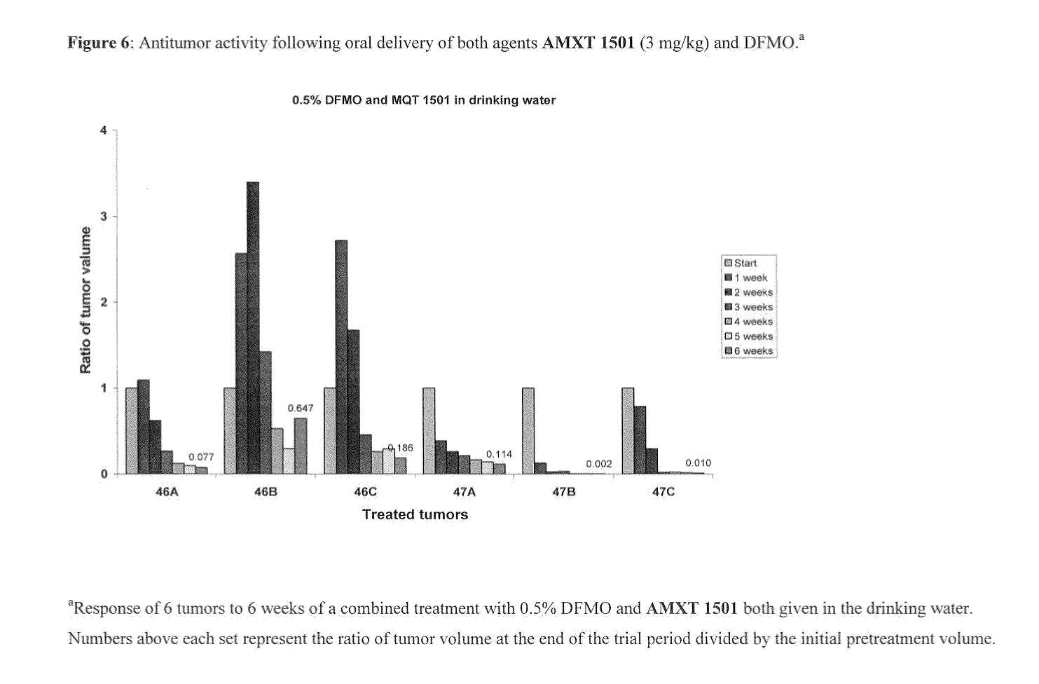

As a further demonstration of the effectiveness of this combination therapy, a second, smaller trial was conducted to assess the efficacy of orally delivered AMXT 1501 on SCC growth (FIG. 6). The concentration of AMXT 1501 was 14 μg/ml, or an average daily dose of ˜50-65 μg (equivalent to ˜3 mg/kg/d). All SCCs responded over a 6-week treatment period, with two tumors exhibiting a complete response (>99% volume reduction). This preliminary result suggests an oral route of administration of polyamine transport inhibitors can be effective. It is expected that great improvements in the oral bioavailability of AMXT 1501 would result from insightful drug formulation.

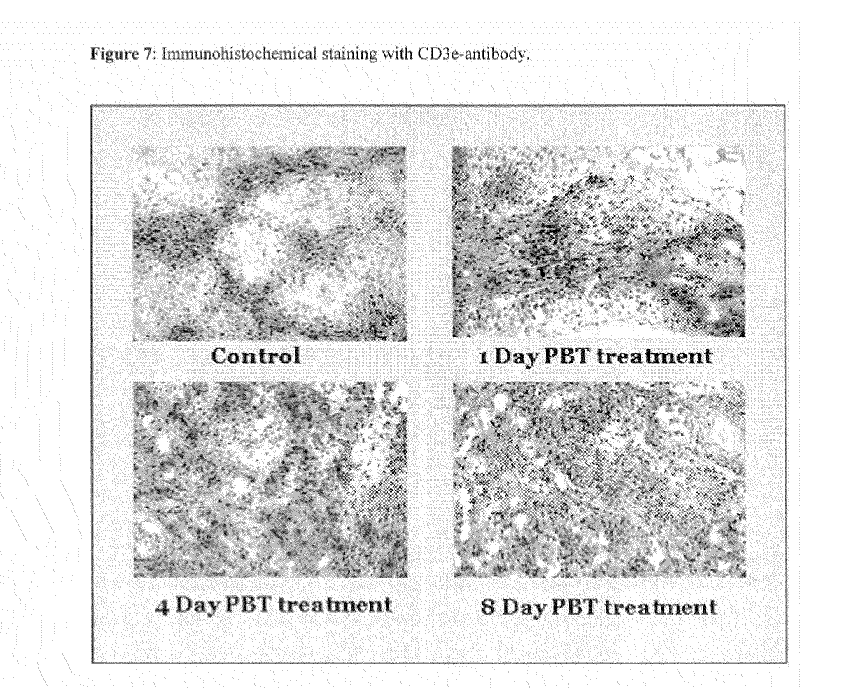

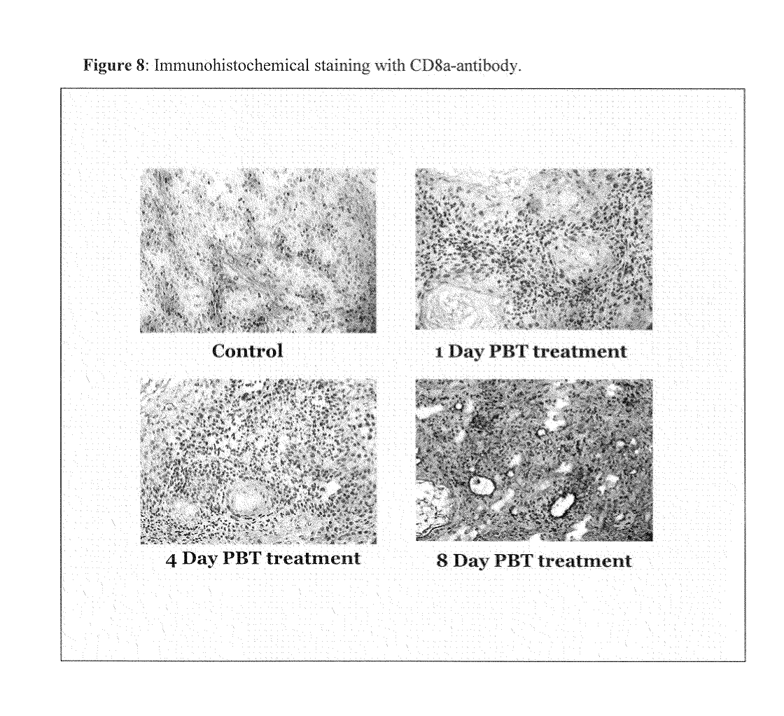

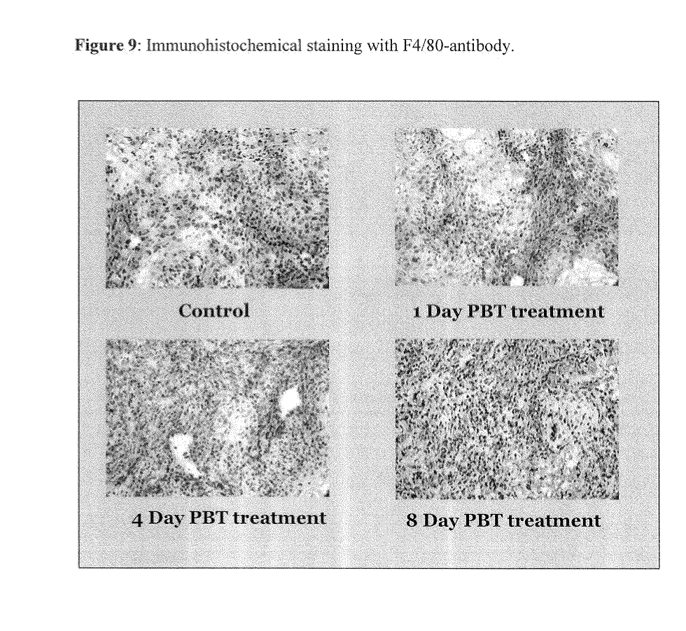

In order to understand the mechanism of polyamine depletion induced tumor regression, we assessed the histological appearance of the tumor and associated stroma in response to combination treatment. We performed immunohistochemical analysis on tumor sections to characterize the cellular nature of the infiltrated stroma cells. Tumor sections were stained with anti-CD3e or anti-CD8a antibodies to identify infiltrating T cells. As shown in FIG. 7, greater CD3e positive cell infiltration was found after treatment, starting as early as 1 day and increasing after 8 days of treatment. A similar phenomenon was also observed for CD8 positive cells. The combination treatment induced a remarkable CD8 infiltration in tumor sections as shown in FIG. 8. We asked whether the combination therapy effected other immune cell infiltration to the tumor. Infiltrating macrophages were detected by staining tumor section with anti-F4/80 antibody. The tumors were gradually infiltrated by greater numbers of macrophages after various treatment lengths (FIG. 9).

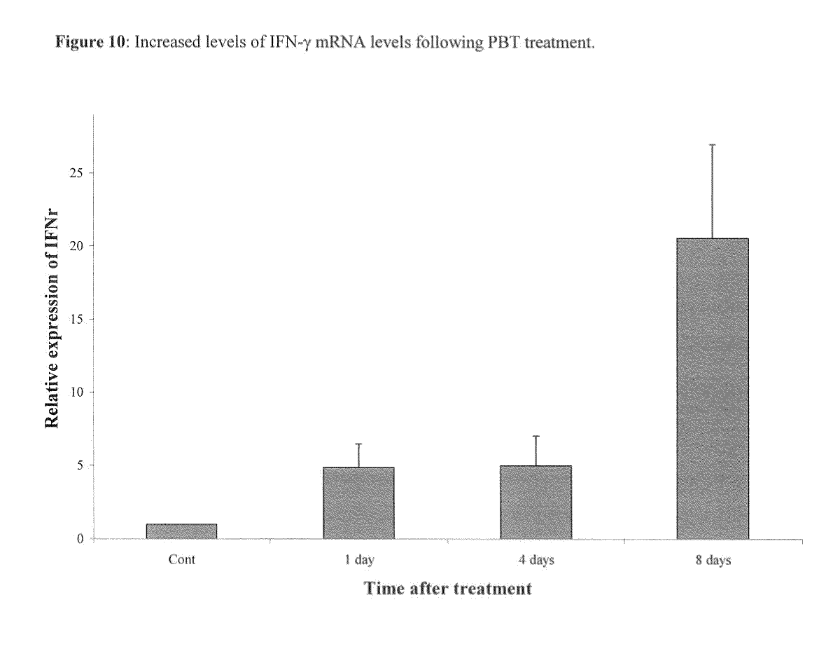

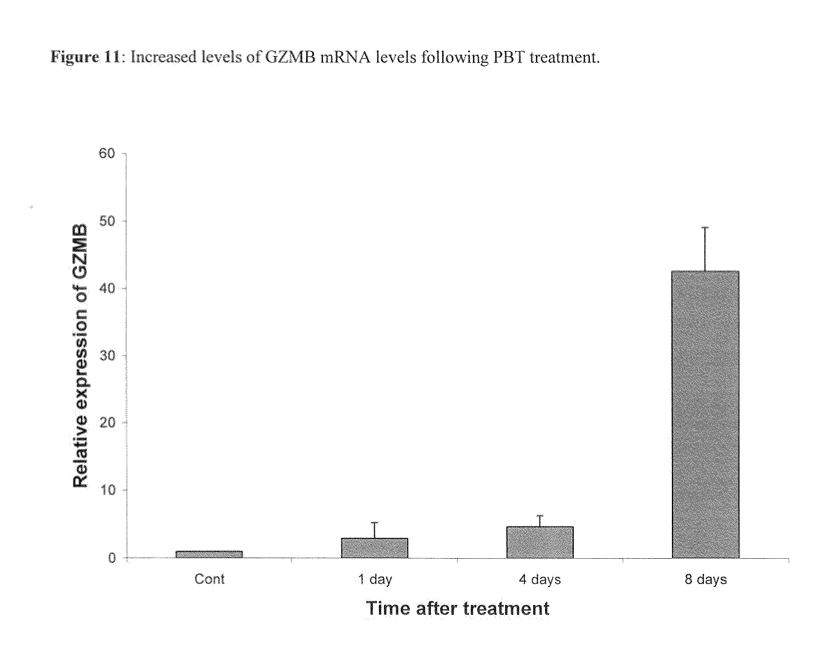

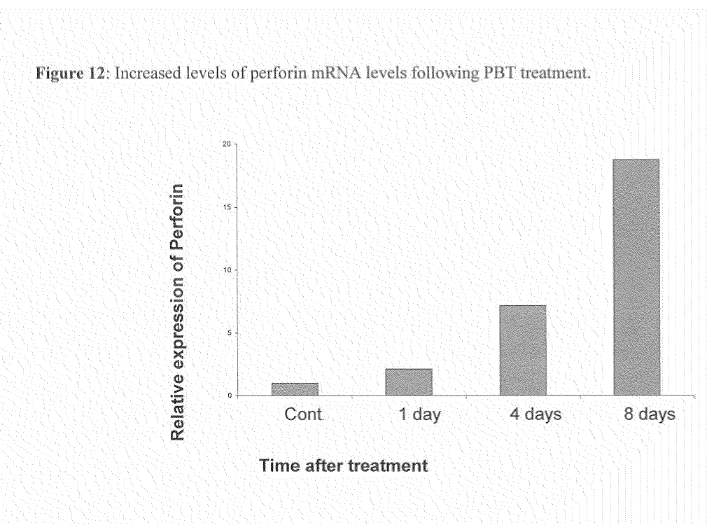

In confirmation of these observations, we performed a real-time PCR technique to determine the effect of the combination therapy on immune cell-mediated cytokines expression. IFN-γ is an important cytokine for cancer therapy, and it is also a marker for T-cell activation and T cell and macrophage interactions. The effect of the combination therapy on tumor IFN-γ expression is shown in FIG. 10. We observed that the combination treatment caused significant increase of IFN-γ mRNA level in tumor as early as 1 day after treatment. After an 8 day treatment, IFN-γ mRNA level in treated tumors was 25 times higher than in control tumors. In addition, a remarkable induction of CD8 T-cell associated cytotoxic molecule (GZMB) expression was also observed in the combination treated tumors and its mRNA levels were significantly elevated after treatment and by 8 days of treatment was elevated 40 fold compared to control (FIG. 11). Finally, data shown in FIG. 12 demonstrate that the expression of perforin is also time-dependently increased following PBT treatment. These results indicate that the PBT therapy caused a rapid response of immune system, including immune cell infiltration as well as immune cell-mediated cytokine mRNA expression levels. It shows that polyamine depletion-induced tumor regression was associated with combined effects on proliferation and immune cell-mediated rejection.

A major theoretical advantage of PBT's potential ability to induce an adaptive immune response would be its long-lived benefit. Drugs or antibodies target one cell at a time and then disappear. Thus, the amount of drugs or antibody has to exceed the number of tumor cells. With PBT's adaptive mechanism, the amount of drug needed is multiplied by the power of the immune system. Furthermore, with targeted drug therapy, the ability of the tumor to overcome the drug's block of a specific pathway is facilitated. With PBT, multiple immunological mechanisms of cancer cell killing are engaged at once. The chance that the tumor will be able to evade such a powerful onslaught is minimized.

The results presented here indicate that PBT can be an effective anti-tumor therapy when used as mono-therapy by itself. Nevertheless, its usefulness may also be expected when used in combination with some of the newer, molecularly targeted, biologic agents such as cetuximab (Erbitux) or bevacizumab (Avastin) now in development for later stage HNSCC tumors.¹⁰⁰ While our results show animal proof-of-concept against squamous cell carcinoma, it is also broadly applicable against a range of epithelial and additional tumor types. Furthermore, the data presented herein show that PBT could be a useful adjuvant for use with other immunotherapeutic drugs being studied or in development now (e.g. IL-12¹⁰¹; IL-15¹⁰² or Anti-IL-10 receptor or Anti-IL-10¹⁰³). The development of immunotherapeutic agents for use against cancer was recently reviewed in a NCI workshop.¹⁰⁴ It would furthermore be expected that the use of PBT as an adjuvant when used with tumor vaccines is warranted.

Cancer in veterinarian animals has become an appealing model for studying cancer in people.¹⁰⁵ Spontaneous HNSCC in felines is considered a predictive natural model for studying treatments for the human disease.¹⁰⁶ We have now demonstrated that PBT treatment shows a positive response in spontaneous HNSCC in domestic cats. In collaboration with Katherine Skorupski, DVM at the University of California at Davis, a Phase I/II dose-escalation trial using oral AMXT 1426 and DFMO was initiated in April, 2007. Thirteen cats with histologically confirmed oral HNSCC were enrolled at UC Davis and their owners delivered the two agents orally at one of three dosages. Two cats were still alive as of January, 2008 with partial responses and with survival times of 132 and 188 days with the overall median survival of 105 days. The general prognosis for these animals is 44 days survival time after diagnosis.¹⁰⁷ Two cats at the highest drug level had reversible vestibular toxicity. It was noted by the veterinarian that it was striking that six cats gained weight during the study period. A manuscript describing these results has been accepted for publication in the Journal of Veterinarian and Comparible Oncology.¹⁰⁸

While not being bound by theory, we can offer several mechanistic hypotheses to explain our results. Firstly, by decreasing the polyamine levels that are normally associated with tumors, we are biochemically unleashing the typically immunosuppressive tumor microenvironment and thus allowing lymphocytes to attack. In addition to that outlined above, a substantial amount of scientific literature data exists, showing that the polyamines, especially spermine, inhibit the immune system's reactivity. We therefore hypothesize that the increased polyamine levels observed associated with cancer cells could be an artifact of the natural selection of those cells. Returning polyamine levels to their normal levels should allow the immune system to recognize the transformed nature of the tumor and allow re-presentation of tumor-associated antigenic peptides. Literature suggests a specific biochemical mechanism by which the polyamine depletion approach could be inducing an adaptive immune response to tumors. It is well established that tumors have associated antigenic molecules. The normal HSP73-TAP mediated translocation of antigenic peptides to the major histocompatibility complex class I molecules for presentation to T cells has been shown to be inhibited by the polyamines.¹⁰⁹ Disruption of the association of HSP73 with TAP has furthermore been shown to be caused by the immunosuppressive polyamine analog deoxymethylsperqualin (MeDSG).^{110,111}

Secondly, we suggest that the polyamine uptake inhibitor drug associated with PBT, AMXT 1501, due to its amphipathic nature may be causing disruption of tumor cells and/or the tumor vascular and the resulting cellular debris is inducing the observed immune activation. The importance of an immunological component of conventional cancer chemotherapy has often been overlooked and agents such as oxaliplatin do indeed cause immune system activation against cancer.^{112, 113} Importantly, it is now understood that necrotic but not apoptotic cell death actually induces the maturation of dendritic cells.¹¹⁴ It is envisioned that a long-lived, and tumor-specific immunotherapy against tumors and their metastases could be developed using Aminex's PBT approach.

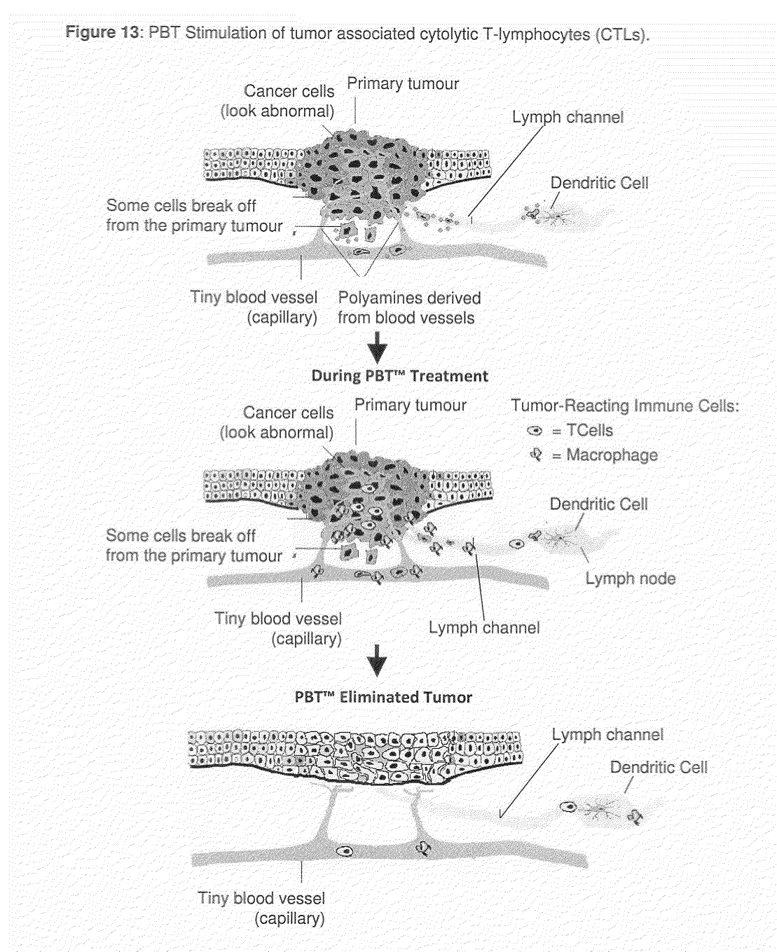

It is certainly predicted that a higher level of AMXT 1501, because of its polyamine-like structure, will be concentrated in DFMO-treated tumors. Higher tumor concentrations of this drug might signal stress and cause cell death, either by apoptosis or necrosis, which allows production of tumor-associated peptide fragments. As outlined by FIG. 13, this process will facilitate an adaptive immune response. Once tumor cell-derived antigenic peptides are presented to mature dendritic cells, long-lived clonogenic, tumor-specific T cells will be produced.

It has recently been recognized that conventional cancer treatments that rely on radiotherapy and chemotherapy exert part of their efficacy by inducing innate and adaptive immune responses. Studies in mice and humans showed that secretion of high-mobility-group box 1 (HMGB1) alarmin protein from dying tumor cells induced an adaptive immunity against tumors by the action of HMGB1 on Toll-like receptor 4 (TLR4) expressed by dendritic cells (DCs).¹¹⁵ This pathway allows efficient cross-presentation of antigens from dying tumors thus initiating an adaptive immunity to cancer. It is important to note that this effect is seen following treatment with most chemotherapeutic agents.¹¹⁶ In therapy damaged or dying cells, HMGB1 dissociates from its normal chromatin cellular partner and is released into the extracellular environment due to loss of plasma membrane integrity. Once outside the cell, HMGB1 acts as a damage-associated molecular pattern molecule that alerts the immune system to the damage. In contrast to apoptotic cell death that are quickly engulfed and cleared by phagocytes, cells dying by a necrotic mechanism release substances that cause immunological response include those causing dendritic cell maturation. This is essential in defending against viral and pathogen infection.¹¹⁷

While the role of HMGB1 in cancer biology is complex, the influence of spermine on this danger signaling biomolecule is well established. Spermine has been shown to inhibit HMGB1-induced inflammatory responses. Tracey has shown the ability of spermine to inhibit endotoxin-mediated immune system activation is mediated through its inhibition of the HMGB1-induced release of several markers of sepsis in mouse models.¹¹⁸ Extensive work has explored the structural features of polyamine analogs that are needed to inhibit the ability of LPS in inducing sepsis in mouse models.¹¹⁹⁻¹²¹ Endotoxin's affects are mediated through the TLR4 receptors, yet when tested, no significant stimulatory effect on human toll-like receptors (TLR) by AMXT 1501 (10 μM) was observed.⁷⁵ This data demonstrated a direct interaction between AMXT 1501 and toll-like receptors is not part of the mechanism of PBT's anticancer effect. Nevertheless, Tracey's work with spermine demonstrates a clear mechanistic connection between the tumor's higher spermine levels and its ability to inhibit the immune-stimulatory effects of HMGB1 protein. It could be that the tumor's propensity to secrete higher concentrations of spermine allow them to hide from the immune system by negating HMGB1 ability to stimulate an immune response. Therefore, the balance between pro-immune effects of HMGB1 and the counter-immune effects of spermine mediated through this pathway may be shifted by using PBT.

The term “comprising” (and its grammatical variations) as used herein is used in the inclusive sense of “having” or “including” and not in the exclusive sense of “consisting only of” The terms “a” and “the” as used herein are understood to encompass the plural as well as the singular.

All publications, patents and patent applications cited in this specification, including the following listing of citations, are herein incorporated by reference, and for any and all purpose, as if each individual publication, patent or patent application were specifically and individually indicated to be incorporated by reference. In the case of inconsistencies, the present disclosure will prevail.

The foregoing description of the disclosure illustrates and describes the present disclosure. Additionally, the disclosure shows and describes only the preferred embodiments but, as mentioned above, it is to be understood that the disclosure is capable of use in various other combinations, modifications, and environments and is capable of changes or modifications within the scope of the concept as expressed herein, commensurate with the above teachings and/or the skill or knowledge of the relevant art.

The embodiments described hereinabove are further intended to explain best modes known of practicing it and to enable others skilled in the art to utilize the disclosure in such, or other, embodiments and with the various modifications required by the particular applications or uses. Accordingly, the description is not intended to limit it to the form disclosed herein. Also, it is intended that the appended claims be construed to include alternative embodiments.

### LISTING OF CITATIONS

1. Gajewski, T. F., Meng, Y., and Harlin, H. (2006) Immune suppression in the tumor microenvironment, J Immunother 29, 233-240.
2. Gajewski, T. F., Meng, Y., Blank, C., Brown, I., Kacha, A., Kline, J., and Harlin, H. (2006) Immune resistance orchestrated by the tumor microenvironment, Immunol Rev 213, 131-145.
3. Algood, H. M., and Cover, T. L. (2006) Helicobacter pylori persistence: an overview of interactions between H. pylori and host immune defenses, Clin Microbiol Rev 19, 597-613.
4. Hajishengallis, G., Wang, M., Liang, S., Shakhatreh, M. A., James, D., Nishiyama, S., Yoshimura, F., and Demuth, D. R. (2008) Subversion of innate immunity by periodontopathic bacteria via exploitation of complement receptor-3, Advances in experimental medicine and biology 632, 203-219.
5. Miller-Kittrell, M., and Sparer, T. E. (2009) Feeling manipulated: cytomegalovirus immune manipulation, Virol J 6, 4.
6. Jonjic, S., Babic, M., Polic, B., and Krmpotic, A. (2008) Immune evasion of natural killer cells by viruses, Curr Opin Immunol 20, 30-38.
7. Krone, B., and Grange, J. M. (2010) Melanoma, Darwinian medicine and the inner world, Journal of cancer research and clinical oncology 136, 1787-1794.
8. Metcalf, B. W., Bey, P., Danzin, C., Jung, M. J., Casara, P., and Vevert, J. P. (1978) Catalytic irreversible inhibition of mammalian ornithine decarboxylase (E.C.4.1.1.17) by substrate and product analogs, Journal of the American Chemical Society 100, 2551-2553.
9. Seiler, N. (2003) Thirty years of polyamine-related approaches to cancer therapy. Retrospect and prospect. Part 2. Structural analogues and derivatives, Curr Drug Targets 4, 565-585.
10. Seiler, N. (2003) Thirty years of polyamine-related approaches to cancer therapy. Retrospect and prospect. Part 1. Selective enzyme inhibitors, Curr Drug Targets 4, 537-564.
11. Ehrlich, P. (1909) Ned Tijdschr beneeskd 5, 273-290.
12. van der Bruggen, P., Traversari, C., Chomez, P., Lurquin, C., De Plaen, E., Van den Eynde, B., Knuth, A., and Boon, T. (1991) A gene encoding an antigen recognized by cytolytic T lymphocytes on a human melanoma, Science 254, 1643-1647.
13. Dunn, G. P., Old, L. J., and Schreiber, R. D. (2004) The Immunobiology of Cancer Immunosurveillance and Immunoediting, Immunity 21, 137-148.
14. Khong, H. T., and Restifo, N. P. (2002) Natural selection of tumor variants in the generation of “tumor escape” phenotypes, Nat Immunol 3, 999-1005.
15. Malmberg, K. J. (2004) Effective immunotherapy against cancer: a question of overcoming immune suppression and immune escape?, Cancer Immunol Immunother 53, 879-892.
16. Zitvogel, L., Apetoh, L., Ghiringhelli, F., and Kroemer, G. (2008) Immunological aspects of cancer chemotherapy, Nat Rev Immunol 8, 59-73.
17. Dunn, G. P., Bruce, A. T., Ikeda, H., Old, L. J., and Schreiber, R. D. (2002) Cancer immunoediting: from immunosurveillance to tumor escape, Nature immunology 3, 991-998.
18. Naito, Y., Saito, K., Shiiba, K., Ohuchi, A., Saigenji, K., Nagura, H., and Ohtani, H. (1998) CD8+ T cells infiltrated within cancer cell nests as a prognostic factor in human colorectal cancer, Cancer research 58, 3491-3494.
19. Mlecnik, B., Tosolini, M., Kirilovsky, A., Berger, A., Bindea, G., Meatchi, T., Bruneval, P., Trajanoski, Z., Fridman, W. H., Pages, F., and Galon, J. (2011) Histopathologic-based prognostic factors of colorectal cancers are associated with the state of the local immune reaction, Journal of clinical oncology: official journal of the American Society of Clinical Oncology 29, 610-618.
20. Finke, L. H., Wentworth, K., Blumenstein, B., Rudolph, N. S., Levitsky, H., and Hoos, A. (2007) Lessons from randomized phase III studies with active cancer immunotherapies—outcomes from the 2006 meeting of the Cancer Vaccine Consortium (CVC), Vaccine 25 Suppl 2, B97-B109.
21. Zou, W. (2006) Regulatory T cells, tumour immunity and immunotherapy, Nature reviews. Immunology 6, 295-307.
22. Banchereau, J., and Steinman, R. M. (1998) Dendritic cells and the control of immunity, Nature 392, 245-252.
23. Kryczek, I., Grybos, M., Karabon, L., Klimczak, A., and Lange, A. (2000) IL-6 production in ovarian carcinoma is associated with histiotype and biological characteristics of the tumour and influences local immunity, British journal of cancer 82, 621-628.
24. Heriot, A. G., Marriott, J. B., Cookson, S., Kumar, D., and Dalgleish, A. G. (2000) Reduction in cytokine production in colorectal cancer patients: association with stage and reversal by resection, British journal of cancer 82, 1009-1012.
25. Rampone, B., Rampone, A., Tirabasso, S., Panariello, S., and Rampone, N. (2001) Immunological variations in women suffering from ovarian cancer. Influence of radical surgical treatment, Minerva Ginecol 53, 116-119.
26. Zou, J. P., Nagata, T., Yamamoto, N., Ono, S., Fujiwara, H., and Hamaoka, T. (1994) Recovery of antitumor CD4+ T cell responsiveness, suppressed in the tumor-bearing state, by release from tumor burden, Journal of cancer research and clinical oncology 120, 279-285.
27. Pegg, A. E., and Feith, D. J. (2007) Polyamines and neoplastic growth, Biochemical Society transactions 35, 295-299.
28. Wallace, H. M., Fraser, A. V., and Hughes, A. (2003) A perspective of polyamine metabolism, The Biochemical journal 376, 1-14.
29. Uehara, N., Shirakawa, S., Uchino, H., and Saeki, Y. (1980) Elevated contents of spermidine and spermine in the erythrocytes of cancer patients, Cancer 45, 108-111.
30. Svensson, K. J., Welch, J. E., Kucharzewska, P., Bengtson, P., Bjurberg, M., Pahlman, S., Ten Dam, G. B., Persson, L., and Belting, M. (2008) Hypoxia-mediated induction of the polyamine system provides opportunities for tumor growth inhibition by combined targeting of vascular endothelial growth factor and ornithine decarboxylase, Cancer research 68, 9291-9301.
31. Cohen, S. S. (1998) A Guide to the Polyamines, Oxford University Press.
32. Igarashi, K., and Kashiwagi, K. (2000) Polyamines: mysterious modulators of cellular functions, Biochemical and biophysical research communications 271, 559-564.
33. Janne, J., Poso, H., and Raina, A. (1978) Polyamines in rapid growth and cancer, Biochimica et biophysica acta 473, 241-293.
34. Scalabrino, G., and Ferioli, M. E. (1981) Polyamines in mammalian tumors. Part I, Advances in cancer research 35, 151-268.
35. Scalabrino, G., and Ferioli, M. E. (1982) Polyamines in mammalian tumors. Part II, Advances in cancer research 36, 1-102.
36. Seiler, N., and Dezeure, F. (1990) Polyamine transport in mammalian cells, The International journal of biochemistry 22, 211-218.
37. Seiler, N., Delcros, J. G., and Moulinoux, J. P. (1996) Polyamine transport in mammalian cells. An update, The international journal of biochemistry & cell biology 28, 843-861.
38. DisPasquale, A., White, D., and McGuire, J. (1978) Epidermal growth factor stimulates putrescine transport and ornithine decarboxylase activity in cultivated human fibroblasts, Experimental cell research 116, 317-323.
39. Pohjanpelto, P. (1976) Putrescine transport is greatly increased in human fibroblasts initiated to proliferate, J Cell Biol 68, 512-520.
40. Volkow, N., Goldman, S. S., Flamm, E. S., Cravioto, H., Wolf, A. P., and Brodie, J. D. (1983) Labeled putrescine as a probe in brain tumors, Science 221, 673-675.
41. Moulinoux, J. P., Quemener, V., and Khan, N. A. (1991) Biological significance of circulating polyamines in oncology, Cell Mol Biol (Noisy-le-grand) 37, 773-783.
42. Sharkis, S. J., Luk, G. D., Collector, M. I., McCann, P. P., Baylin, S. B., and Sensenbrenner, L. L. (1983) Regulation of hematopoiesis II: the role of polyamine inhibition on helper or suppressor influences of the thymus, Blood 61, 604-607.
43. Niskanen, E., Kallio, A., McCann, P. P., and Baker, D. G. (1983) The role of polyamine biosynthesis in hematopoietic precursor cell proliferation in mice, Blood 61, 740-745.
44. Jaenne, J., Raina, A., and Siimes, M. (1964) Spermidine and Spermine in Rat Tissues at Different Ages, Acta Physiol Scand 62, 352-358.
45. Tabor, H., and Tabor, C. W. (1964) Spermidine, Spermine, and Related Amines, Pharmacol Rev 16, 245-300.
46. Byrd, W. J., Jacobs, D. M., and Amoss, M. S. (1977) Synthetic polyamines added to cultures containing bovine sera reversibly inhibit in vitro parameters of immunity, Nature 267, 621-623.
47. Van Pel, A., and Boon, T. (1982) Protection against a nonimmunogenic mouse leukemia by an immunogenic variant obtained by mutagenesis, Proceedings of the National Academy of Sciences of the United States of America 79, 4718-4722.
48. Waldmann, T. A. (2003) Immunotherapy: past, present and future, Nat Med 9, 269-277.
49. Boon, T., Coulie, P. G., and Van den Eynde, B. (1997) Tumor antigens recognized by T cells, Immunol Today 18, 267-268.
50. Chamaillard, L., Catros-Quemener, V., Delcros, J. G., Bansard, J. Y., Havouis, R., Desury, D., Commeurec, A., Genetet, N., and Moulinoux, J. P. (1997) Polyamine deprivation prevents the development of tumour-induced immune suppression, British journal of cancer 76, 365-370.
51. Chamaillard, L., Quemener, V., Havouis, R., and Moulinoux, J. P. (1993) Polyamine deprivation stimulates natural killer cell activity in cancerous mice, Anticancer research 13, 1027-1033.
52. Kano, Y., Soda, K., Nakamura, T., Saitoh, M., Kawakami, M., and Konishi, F. (2007) Increased blood spermine levels decrease the cytotoxic activity of lymphokine-activated killer cells: a novel mechanism of cancer evasion, Cancer immunology, immunotherapy: CII 56, 771-781.
53. Bowlin, T. L., McKown, B. J., Davis, G. F., and Sunkara, P. S. (1986) Effect of polyamine depletion in vivo by DL-alpha-difluoromethylornithine on functionally distinct populations of tumoricidal effector cells in normal and tumor-bearing mice, Cancer research 46, 5494-5498.
54. Bowlin, T. L., Hoeper, B. J., Rosenberger, A. L., Davis, G. F., and Sunkara, P. S. (1990) Effects of three irreversible inhibitors of ornithine decarboxylase on macrophage-mediated tumoricidal activity and antitumor activity in B16F1 tumor-bearing mice, Cancer research 50, 4510-4514.
55. Flescher, E., Bowlin, T. L., and Talal, N. (1989) Polyamine oxidation down-regulates IL-2 production by human peripheral blood mononuclear cells, Journal of immunology 142, 907-912.
56. Gensler, H. L. (1991) Prevention by alpha-difluoromethylornithine of skin carcinogenesis and immunosuppression induced by ultraviolet irradiation, Journal of cancer research and clinical oncology 117, 345-350.
57. Gervais, A., Leveque, J., Bouet-Toussaint, F., Burtin, F., Lesimple, T., Sulpice, L., Patard, J. J., Genetet, N., and Catros-Quemener, V. (2005) Dendritic cells are defective in breast cancer patients: a potential role for polyamine in this immunodeficiency, Breast Cancer Res 7, R326-335.
58. Evans, C. H., Lee, T. S., and Flugelman, A. A. (1995) Spermine-directed immunosuppression of cervical carcinoma cell sensitivity to a majority of lymphokine-activated killer lymphocyte cytotoxicity, Nat Immun 14, 157-163.
59. Zhang, M., Caragine, T., Wang, H., Cohen, P. S., Botchkina, G., Soda, K., Bianchi, M., Ulrich, P., Cerami, A., Sherry, B., and Tracey, K. J. (1997) Spermine inhibits proinflammatory cytokine synthesis in human mononuclear cells: a counterregulatory mechanism that restrains the immune response, J Exp Med 185, 1759-1768.
60. Zhang, M., Borovikova, L. V., Wang, H., Metz, C., and Tracey, K. J. (1999) Spermine inhibition of monocyte activation and inflammation, Mol Med 5, 595-605.
61. Gervais, A., Bouet-Toussaint, F., Toutirais, 0., De La Pintiere, C. T., Genetet, N., and Catros-Quemener, V. (2005) Ex vivo expansion of antitumor cytotoxic lymphocytes with tumor-associated antigen-loaded dendritic cells, Anticancer research 25, 2177-2185.
62. Susskind, B. M., and Chandrasekaran, J. (1987) Inhibition of cytolytic T lymphocyte maturation with ornithine, arginine, and putrescine, Journal of immunology 139, 905-912.
63. Szabo, C., Southan, G. J., Thiemermann, C., and Vane, J. R. (1994) The mechanism of the inhibitory effect of polyamines on the induction of nitric oxide synthase: role of aldehyde metabolites, Br J Pharmacol 113, 757-766.
64. Bussiere, F. I., Chaturvedi, R., Cheng, Y., Gobert, A. P., Asim, M., Blumberg, D. R., Xu, H., Kim, P. Y., Hacker, A., Casero, R. A., Jr., and Wilson, K. T. (2005) Spermine causes loss of innate immune response to Helicobacter pylori by inhibition of inducible nitric-oxide synthase translation, The Journal of biological chemistry 280, 2409-2412.
65. Chaturvedi, R., Cheng, Y., Asim, M., Bussiere, F. I., Xu, H., Gobert, A. P., Hacker, A., Casero, R. A., Jr., and Wilson, K. T. (2004) Induction of polyamine oxidase 1 by Helicobacter pylori causes macrophage apoptosis by hydrogen peroxide release and mitochondrial membrane depolarization, The Journal of biological chemistry 279, 40161-40173.
66. Shirahata, A., and Pegg, A. E. (1985) Regulation of S-adenosylmethionine decarboxylase activity in rat liver and prostate, The Journal of biological chemistry 260, 9583-9588.
67. Gobert, A. P., McGee, D. J., Akhtar, M., Mendz, G. L., Newton, J. C., Cheng, Y., Mobley, H. L., and Wilson, K. T. (2001) Helicobacter pylori arginase inhibits nitric oxide production by eukaryotic cells: a strategy for bacterial survival, Proceedings of the National Academy of Sciences of the United States of America 98, 13844-13849.
68. Flescher, E., Bowlin, T. L., and Talal, N. (1992) Regulation of IL-2 production by mononuclear cells from rheumatoid arthritis synovial fluids, Clin Exp Immunol 87, 435-437.
69. Flescher, E., Bowlin, T. L., Ballester, A., Houk, R., and Talal, N. (1989) Increased polyamines may downregulate interleukin 2 production in rheumatoid arthritis, The Journal of clinical investigation 83, 1356-1362.
70. Bowlin, T. L., Prakash, N. J., Edwards, M. L., and Sjoerdsma, A. (1991) Participation of T-lymphocytes in the curative effect of a novel synthetic polyamine analogue, N,N′-bis[3-(ethylamino)propyl]-1,7-heptanediamine, against L1210 leukemia in vivo, Cancer research 51, 62-66.
71. Prakash, N. J., Bowlin, T. L., Edwards, M. L., Sunkara, P. S., and Sjoerdsma, A. (1990) Antitumor activity of a novel synthetic polyamine analogue, N,N′-bis-[3-(ethylamino)-propyl]-1-7-heptane diamine: potentiation by polyamine oxidase inhibitors, Anticancer research 10, 1281-1287.
72. Umezawa, H., Nishikawa, K., Shibasaki, C., Takahashi, K., Nakamura, T., and Takeuchi, T. (1987) Involvement of cytotoxic T-lymphocytes in the antitumor activity of spergualin against L1210 cells, Cancer research 47, 3062-3065.
73. Broxmeyer, H. E., Orschell, C. M., Clapp, D. W., Hangoc, G., Cooper, S., Plett, P. A., Liles, W. C., Li, X., Graham-Evans, B., Campbell, T. B., Calandra, G., Bridger, G., Dale, D. C., and Srour, E. F. (2005) Rapid mobilization of murine and human hematopoietic stem and progenitor cells with AMD3100, a CXCR4 antagonist, J Exp Med 201, 1307-1318.
74. Smith, M. C., Luker, K. E., Garbow, J. R., Prior, J. L., Jackson, E., Piwnica-Worms, D., and Luker, G. D. (2004) CXCR4 regulates growth of both primary and metastatic breast cancer, Cancer research 64, 8604-8612.
75. Schaefer, L., Babelova, A., Kiss, E., Hausser, H. J., Baliova, M., Krzyzankova, M., Marsche, G., Young, M. F., Mihalik, D., Gotte, M., Malle, E., Schaefer, R. M., and Grone, H. J. (2005) The matrix component biglycan is proinflammatory and signals through Toll-like receptors 4 and 2 in macrophages, The Journal of clinical investigation 115, 2223-2233.
76. Bitonti, A. J., McCann, P. P., and Sjoerdsma, A. (1986) Necessity of antibody response in the treatment of African trypanosomiasis with alpha-difluoromethylornithine, Biochemical pharmacology 35, 331-334.
77. Greenwood, B. M., Whittle, H. C., and Molyneux, D. H. (1973) Immunosuppression in Gambian trypanosomiasis, Trans R Soc Trop Med Hyg 67, 846-850.
78. Rurangirwa, F. R., Tabel, H., Losos, G. J., and Tizard, I. R. (1979) Suppression of antibody response to Leptospira biflexa and Brucella abortus and recovery from immunosuppression after Berenil treatment, Infect Immun 26, 822-826.
79. Francois, G., Van Looveren, M., and Timperman, G. (1997) Evaluation of alpha-difluoromethylornithine (DFMO) as a tool to induce protective immunity against Plasmodium berghei malaria, Ann Trop Med Parasitol 91, 103-106.
80. Gillet, J., Bone, G., Lowa, P., Charlier, J., Rona, A. M., and Schechter, P. J. (1986) alpha-Difluoromethylornithine induces protective immunity in mice inoculated with Plasmodium berghei sporozoites, Trans R Soc Trop Med Hyg 80, 236-239.
81. Tyms, A. S., and Williamson, J. D. (1982) Inhibitors of polyamine biosynthesis block human cytomegalovirus replication, Nature 297, 690-691.
82. Tyms, A. S., and Williamson, J. D. (1980) Polyamine metabolism in MRC-5 cells infected with human cytomegalovirus, J Gen Virol 48, 183-191.
83. Tyms, A. S., Scamans, E., and Williamson, J. D. (1979) Polyamine metabolism in MRCS cells infected with different herpesviruses, Biochemical and biophysical research communications 86, 312-318.
84. Clarke, J. R., and Tyms, A. S. (1991) Polyamine biosynthesis in cells infected with different clinical isolates of human cytomegalovirus, J Med Virol 34, 212-216.
85. Colombatto, S., De Agostini, M., Corsi, D., and Sinicco, A. (1989) Polyamines in lymphocytes from patients infected by human immunodeficiency virus, Biol Chem Hoppe Seyler 370, 745-748.
86. Don, S., Wiener, H., and Bachrach, U. (1975) Specific increase in polyamine levels in chick embryo cells transformed by Rous sarcoma virus, Cancer research 35, 194-198.
87. Holtta, E., Vartio, T., Janne, J., Vaheri, A., and Hovi, T. (1981) Temperature-dependent and transformation-associated changes in polyamine metabolism in normal and Rous sarcoma virus-infected chick embryo fibroblasts, Biochimica et biophysica acta 677, 1-6.
88. White, E. L., Rose, L. M., Allan, P. W., Buckheit, R. W., Jr., Shannon, W. M., and Secrist, J. A., 3rd. (1998) Polyamine pools in HIV-infected cells, J Acquir Immune Defic Syndr Hum Retrovirol 17, 101-103.
89. Gibson, W., van Breemen, R., Fields, A., LaFemina, R., and Irmiere, A. (1984) D,L-alpha-difluoromethylornithine inhibits human cytomegalovirus replication, J Virol 50, 145-154.
90. Galliani, G., Colombo, G., and Luzzani, F. (1983) Contragestational effects of DL-alpha-difluoro-methylornithine, an irreversible inhibitor of ornithine decarboxylase, in the hamster, Contraception 28, 159-170.
91. Reddy, P. R., and Rukmini, V. (1981) Alpha-difluoromethylornithine as a postcoitally effective antifertility agent in female rats, Contraception 24, 215-221.
92. Fozard, J. R., Part, M. L., Prakash, N. J., Grove, J., Schechter, P. J., Sjoerdsma, A., and Koch-Weser, J. (1980) L-Ornithine decarboxylase: an essential role in early mammalian embryogenesis, Science 208, 505-508.
93. Vallely, P. J., Sharrard, R. M., and Rees, R. C. (1988) The identification of factors in seminal plasma responsible for suppression of natural killer cell activity, Immunology 63, 451-456.
94. Clark, D. A., Delcros, J. G., and Craft, I. (1996) High levels of spermine in IVF medium as a negative predictor of subsequent success of embryo transfer, J Assist Reprod Genet 13, 464-467.
95. Fernandez, C., Sharrard, R. M., Talbot, M., Reed, B. D., and Monks, N. (1995) Evaluation of the significance of polyamines and their oxidases in the aetiology of human cervical carcinoma, British journal of cancer 72, 1194-1199.
96. Tannehill-Gregg, S. H., Levine, A. L., and Rosol, T. J. (2006) Feline head and neck squamous cell carcinoma: a natural model for the human disease and development of a mouse model, Veterinary and Comparative Oncology 4, 84-97.
97. Burns, M. R., Graminski, G. F., Weeks, R. S., Chen, Y., and O'Brien, T. G. (2009) Lipophilic lysine-spermine conjugates are potent polyamine transport inhibitors for use in combination with a polyamine biosynthesis inhibitor, Journal of medicinal chemistry 52, 1983-1993.
98. Megosh, L., Gilmour, S. K., Rosson, D., Soler, A. P., Blessing, M., Sawicki, J. A., and O'Brien, T. G. (1995) Increased frequency of spontaneous skin tumors in transgenic mice which overexpress ornithine decarboxylase, Cancer research 55, 4205-4209.
99. O'Brien, T. G., Megosh, L. C., Gilliard, G., and Soler, A. P. (1997) Ornithine decarboxylase overexpression is a sufficient condition for tumor promotion in mouse skin, Cancer research 57, 2630-2637.
100. Gold, K. A., Lee, H. Y., and Kim, E. S. (2009) Targeted therapies in squamous cell carcinoma of the head and neck, Cancer 115, 922-935.
101. Gollob, J. A., Mier, J. W., Veenstra, K., McDermott, D. F., Clancy, D., Clancy, M., and Atkins, M. B. (2000) Phase I trial of twice-weekly intravenous interleukin 12 in patients with metastatic renal cell cancer or malignant melanoma: ability to maintain IFN-gamma induction is associated with clinical response, Clinical cancer research: an official journal of the American Association for Cancer Research 6, 1678-1692.
102. Klebanoff, C. A., Finkelstein, S. E., Surman, D. R., Lichtman, M. K., Gattinoni, L., Theoret, M. R., Grewal, N., Spiess, P. J., Antony, P. A., Palmer, D. C., Tagaya, Y., Rosenberg, S. A., Waldmann, T. A., and Restifo, N. P. (2004) IL-15 enhances the in vivo antitumor activity of tumor-reactive CD8+ T cells, Proceedings of the National Academy of Sciences of the United States of America 101, 1969-1974.
103. Taylor, A., Verhagen, J., Blaser, K., Akdis, M., and Akdis, C. A. (2006) Mechanisms of immune suppression by interleukin-10 and transforming growth factor-beta: the role of T regulatory cells, Immunology 117, 433-442.
104. http://dcb.nih.gov/ImmmunAgentWork/Filan_NCI_Workshop_Proceedings_{—}23Oct07.pdf
105. Gordon, I., Paoloni, M., Mazcko, C., and Khanna, C. (2009) The Comparative Oncology Trials Consortium: using spontaneously occurring cancers in dogs to inform the cancer drug development pathway, PLoS Med 6, e1000161.
106. Tannehill-Gregg, S. H., Levine, A. L., and Rosol, T. J. (2006) Feline head and neck squamous cell carcinoma: a natural model for the human disease and development of a mouse model, Vet Comp Oncol 4, 84-97.
107. Hayes, A. M., Adams, V. J., Scase, T. J., and Murphy, S. (2007) Survival of 54 cats with oral squamous cell carcinoma in United Kingdom general practice, J Small Anim Pract 48, 394-399.
108. Skorupski, K. A., O'Brien, T. G., Guerrero, T., Rodriguez, C. O., and Burns, M. R. (2011) Phase I/II clinical trial of 2-difluoromethylornithine (DFMO) and a novel polyamine transport inhibitor (MQT 1426) for feline oral squamous cell carcinoma., Vet Comp Oncol 9.
109. Ohshima, S. T., T.; Yoshida, H.; Kamiguchi, K.; Fujiwara, O.; Tamura, T.; Hirohashi, Y.; Sahara, H.; Nakagawa, T.; Sato, B. N. (2000) Intracellular polyamine regulates MHC class I antigen presentation by affecting the interaction between 73 kD heat shock protein and TAPI, Cell Stress & Chaperones 5, 391.
110. Sugawara, A., Torigoe, T., Tamura, Y., Kamiguchi, K., Nemoto, K., Oguro, H., and Sato, N. (2009) Polyamine compound deoxyspergualin inhibits heat shock protein-induced activation of immature dendritic cells, Cell Stress Chaperones 14, 133-139.
111. Kamiguchi, K., Torigoe, T., Fujiwara, 0., Ohshima, S., Hirohashi, Y., Sahara, H., Hirai, I., Kohgo, Y., and Sato, N. (2008) Disruption of the association of 73 kDa heat shock cognate protein with transporters associated with antigen processing (TAP) decreases TAP-dependent translocation of antigenic peptides into the endoplasmic reticulum, Microbiol Immunol 52, 94-106.
112. Aymeric, L., Apetoh, L., Ghiringhelli, F., Tesniere, A., Martins, I., Kroemer, G., Smyth, M. J., and Zitvogel, L. (2010) Tumor cell death and ATP release prime dendritic cells and efficient anticancer immunity, Cancer research 70, 855-858.
113. Tesniere, A., Schlemmer, F., Boige, V., Kepp, O., Martins, I., Ghiringhelli, F., Aymeric, L., Michaud, M., Apetoh, L., Barault, L., Mendiboure, J., Pignon, J. P., Jooste, V., van Endert, P., Ducreux, M., Zitvogel, L., Piard, F., and Kroemer, G. (2010) Immunogenic death of colon cancer cells treated with oxaliplatin, Oncogene 29, 482-491.
114. Sauter, B., Albert, M. L., Francisco, L., Larsson, M., Somersan, S., and Bhardwaj, N. (2000) Consequences of cell death: exposure to necrotic tumor cells, but not primary tissue cells or apoptotic cells, induces the maturation of immunostimulatory dendritic cells, J Exp Med 191, 423-434.
115. Apetoh, L., Ghiringhelli, F., Tesniere, A., Obeid, M., Ortiz, C., Criollo, A., Mignot, G., Maiuri, M. C., Ullrich, E., Saulnier, P., Yang, H., Amigorena, S., Ryffel, B., Barrat, F. J., Saftig, P., Levi, F., Lidereau, R., Nogues, C., Mira, J. P., Chompret, A., Joulin, V., Clavel-Chapelon, F., Bourhis, J., Andre, F., Delaloge, S., Tursz, T., Kroemer, G., and Zitvogel, L. (2007) Toll-like receptor 4-dependent contribution of the immune system to anticancer chemotherapy and radiotherapy, Nat Med 13, 1050-1059.
116. Apetoh, L., Ghiringhelli, F., Tesniere, A., Criollo, A., Ortiz, C., Lidereau, R., Mariette, C., Chaput, N., Mira, J. P., Delaloge, S., Andre, F., Tursz, T., Kroemer, G., and Zitvogel, L. (2007) The interaction between HMGB1 and TLR4 dictates the outcome of anticancer chemotherapy and radiotherapy, Immunol Rev 220, 47-59.
117. Zong, W. X., and Thompson, C. B. (2006) Necrotic death as a cell fate, Genes Dev 20, 1-15.
118. Zhu, S., Ashok, M., Li, J., Li, W., Yang, H., Wang, P., Tracey, K. J., Sama, A. E., and Wang, H. (2009) Spermine protects mice against lethal sepsis partly by attenuating surrogate inflammatory markers, Mol Med 15, 275-282.
119. Burns, M. R., Jenkins, S. A., Kimbrell, M. R., Balakrishna, R., Nguyen, T. B., Abbo, B. G., and David, S. A. (2007) Polycationic sulfonamides for the sequestration of endotoxin, Journal of medicinal chemistry 50, 877-888.
120. Burns, M. R., Jenkins, S. A., Wood, S. J., Miller, K., and David, S. A. (2006) Structure-activity relationships in lipopolysaccharide neutralizers: design, synthesis, and biological evaluation of a 540-membered amphipathic bisamide library, Journal of combinatorial chemistry 8, 32-43.
121. Burns, M. R., Wood, S. J., Miller, K. A., Nguyen, T., Cromer, J. R., and David, S. A. (2005) Lysine-spermine conjugates: hydrophobic polyamine amides as potent lipopolysaccharide sequestrants, Bioorganic & medicinal chemistry 13, 2523-2536.

What is claimed as new and desired to be protected by Letters Patent of the United States is:

1. A method of reducing the suppression of an immune reaction in a disease state comprising the administration of a polyamine biosynthesis inhibitor and a polyamine transport inhibitor to a patient in need thereof.

2. A method of reducing the suppression of an immune reaction in a disease state comprising administering an antizyme inducer to a patient in need thereof.

3. A method of reducing the suppression of an immune reaction in a disease state comprising administering a polyamine sulfonamide to a patient in need thereof.

4. A method of administering a combination therapy comprising administering a polyamine transport inhibitor and difluoromethylornithine to a patient receiving tumor therapy.

5. A method of administering a combination tumor-directed immunotherapeutic therapy comprising administering a polyamine transport inhibitor and difluoromethylornithine to a patient receiving Vascular Endothelial Growth Factor (VEGF)-targeting chemotherapeutic drugs.

6. The method of claim 1, wherein the polyamine biosynthesis inhibitor is difluoromethylornithine (DFMO).

7. The method of claim 1, wherein the polyamine biosynthesis inhibitor is SAM486A.

8. The method of claim 1, wherein the polyamine transport inhibitor is AMXT 1426.

9. The method of claim 1, wherein the polyamine transport inhibitor is AMXT 1501.

10. The method of claim 1, wherein the polyamine transport inhibitor is AMXT 1569.

11. The method of claim 1, wherein the polyamine transport inhibitor is AMXT 1505.

12. The method of claim 1, wherein the disease state is cancer.

13. The method of claim 1, wherein the disease state is gastric cancer.

14. The method of claim 1, wherein the disease state is malaria.

15. The method of claim 1, further comprising improving the efficacy of immunotherapy of cancer.

16. The method of claim 1, further comprising improving the efficacy of a vaccine therapy against the disease state.

17. The method of claim 1, further comprising improving the efficacy of a vaccine therapy against cancer.

18. The method of claim 1, further comprising improving the efficacy of a vaccine therapy against HIV infection.

19. The method of claim 1, further comprising improving the efficacy of a vaccine therapy against influenza.

20. The method of claim 1, further comprising improving the efficacy of a vaccine therapy against a bacterial infection.

21. A composition comprising:
a polyamine biosynthesis inhibitor or a derivative thereof;
a polyamine transport inhibitor or a derivative thereof; and
at least one of an excipient, a diluent, and a vehicle.

22. The composition of claim 21, wherein the at least one of an excipient, a diluent, and a vehicle is pharmaceutically or cosmetically acceptable.

23. The composition of claim 21, wherein the at least one of an excipient, a diluent, and a vehicle is for topical or intra-aural administration.

24. The composition of claim 21, formulated for intravenous, subcutaneous, intramuscular, intracranial, intraperitoneal, topical, transdermal, intravaginal, intranasal, intrabronchial, intraocular, intraaural, rectal, or parenteral administration.

25. A method of guiding an anticancer treatment comprising measuring the presence of a functional immune system in a patient receiving the anticancer treatment.

26. A method of preventing peptic ulcers comprising administering the composition according to claim 21.

27. A method of preventing a pregnancy comprising inducing an immune response to sperm.

28. The method of claim 8, wherein AMXT 1426 is administered at a dose of about 1.4 to 200 mg/kg/d.

29. The method of claim 9, wherein AMXT 1501 is administered at a dose of about 0.1 to 100 mg/kg/d.
